(19) United States
(12) Patent Application Publication
(10) Pub. No.: US 2025/0009285 A1
(43) Pub. Date: Jan. 9, 2025

(54) PRE-SEIZURE DETECTION DEVICE INCORPORATING ELECTRONIC NOSE TECHNOLOGY

(71) Applicant: University of Georgia Research Foundation, Inc., Athens, GA (US)

(72) Inventor: Ashley Morgan Galanti, Athens, GA (US)

(21) Appl. No.: 18/752,890

(22) Filed: Jun. 25, 2024

Related U.S. Application Data
(60) Provisional application No. 63/511,221, filed on Jun. 30, 2023.

(51) Int. Cl. A61B 5/00; A61B 5/01; A61B 5/0205; A61B 5/024; A61B 5/0531; A61B 5/08; A61B 5/11; A61B 5/145

(52) U.S. Cl. CPC A61B 5/4094; A61B 5/0205; A61B 5/082; A61B 5/14517; A61B 5/14546; A61B 5/681; A61B 5/6822; A61B 5/6833; G01N 33/4975; A61B 5/01; A61B 5/024; A61B 5/0531; A61B 5/11; A61B 2560/0462; A61B 2562/0219; A61B 2562/0285; A61B 2562/043; A61B 2562/125

(57) ABSTRACT

In one aspect, the disclosure relates to a sensor for predicting epileptic seizures in a subject. In an aspect, the sensor includes carbon nanotubes (CNTs) functionalized with organic molecules that can reversibly bind pre-seizure volatile organic compounds (VOCs) by IT-IT bonding, polar bonding, van der Waals interactions, and the like. The sensor can detect the pre-seizure VOCs from biological fluids including sweat, saliva, and breath, thus allowing the prediction of a seizure from 10 to 45 minutes before the seizure occurs, allowing a subject wearing the sensor to take actions to ensure safety and/or to alert caregivers. In an aspect, the sensor can be incorporated into a wearable device that also monitors vital signs such as heart rate, body temperature, skin impedance, and/or body movements in order to predict the type of epileptic seizure. Also disclosed are methods of making the sensors and methods for predicting epileptic seizures.

### CROSS REFERENCE TO RELATED APPLICATIONS

This application claims the benefit of U.S. Provisional Application No. 63/511,221 filed on Jun. 30, 2023, which is incorporated herein by reference in its entirety.

### BACKGROUND

Epilepsy affects 65 million people around the world, with 3.4 million of those living in the United States. Generalized epilepsy episodes can be divided into absence seizures and tonic-clonic seizures and accounts for about 30% of cases and occurs in multiple parts of the brain. Subjects experiencing absence seizures typically experience a few seconds of staring and/or rapid blinking, while subjects experiencing tonic-clonic seizures experience convulsions and/or loss of consciousness. Focal or partial epilepsy accounts for about 70% of cases and occurs in one part of the brain. Focal or partial epilepsy episodes can be categorized as simple, complex, or secondary generalized. Subjects experiencing simple seizures may experience twitching or changes in sensation, while subjects experiencing complex seizures may exhibit confusion and/or be unable to respond to questions or directions for up to several minutes. Meanwhile, secondary generalized seizures starts out as focal epilepsy and switches to generalized epilepsy in another area of the brain.

One third of patients fail to achieve seizure control through anti-epileptic drugs and over 50% are not candidates for corrective surgery. For others, surgery is inaccessible due to geographical location, lack of medical insurance, and other factors. Up to 50,000 deaths in the United States each year can be attributed to or associated with epilepsy including, but not limited to, sudden unexpected death in epilepsy (SUDEP), status epilepticus, aspiration pneumonia, and suicide, while accidental deaths and injuries stemming from epileptic seizures may include drowning, motor vehicle accidents, and the like. Head injury, dental injury, burns, water immersion, and fracture are all risks that may result from an epileptic seizure occurring during everyday activities. As a result, patients with epilepsy have mortality rates 4-7 times higher than those of the general population.

Current strategies for assisting epileptic patients manage or predict seizures include motion detectors, heart rate monitors, smart watches, and surgically-implanted vagus nerve stimulation devices. However, each of these devices has drawbacks. Motion detectors are typically attached to a mattress and send an alert through a speaker to others in the home. However, as they monitor random movements during sleep, they can have an 89% false positive rate and may only give warning seconds prior to a seizure. Heart rate monitors and smart watches can alert the patient and emergency contacts through an app, but have about a 75% false positive rate and only warn seconds prior. Vagus nerve stimulation (VNS) devices are surgically implanted. They connect to the vagus nerve and send an electrical pulse to the brain when a potential seizure is detected. However, VNS devices are invasive to implant, costly, and still suffer from a 60% false positive rate. In some aspects, devices may only detect or predict one type of seizure (e.g., focal) though an epileptic individual can experience 20-30 different seizures per day of 4-5 different types.

Pre-seizure biomarkers have been identified through analysis of saliva, sweat, and breath of a sample population of epileptic individuals during the ictal period at the start of a seizure. These represent 9 volatile organic compounds (VOCs):

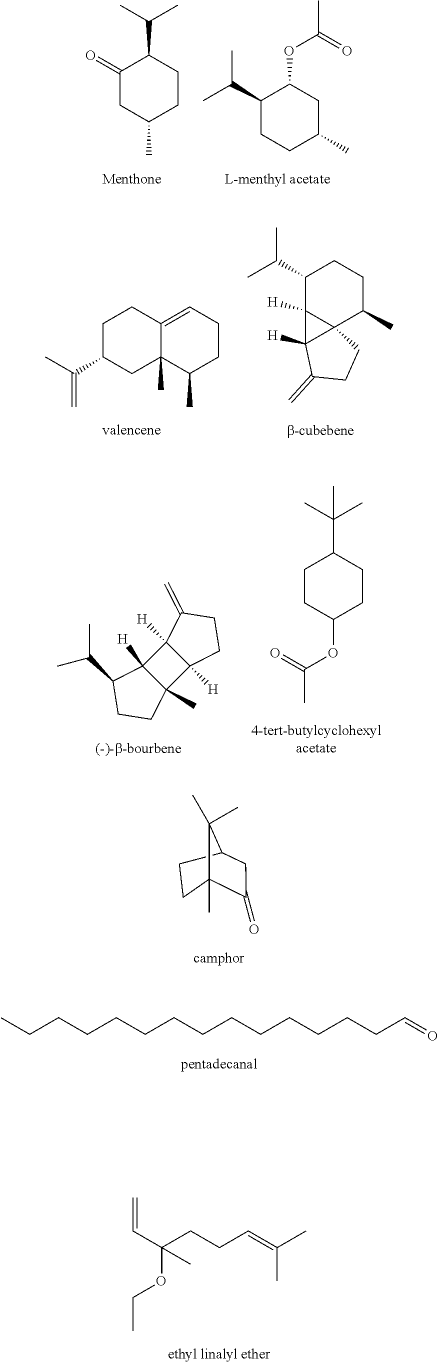

In some cases, patients may have seizure alert dogs. Seizure alert dogs offer a 95% accuracy rate by detecting the VOCs emitted from the body 10-45 minutes before a seizure occurs and alerting the owner through a trained action. However, training and veterinary expenses can be costly, and patients may spend 10 years or more on a waiting list before a dog becomes available.

Early detection of epilepsy and/or other diseases could allow for early intervention and would lead to fewer preventable deaths. In the case of epilepsy, a pre-seizure alert could allow someone to get to a safe space or take emergency medication. Epilepsy also carries a social stigma due to the current unpredictability of seizures. Epileptic individuals may suffer discrimination in school environments and may not be able to maintain certain jobs, which can lead to feelings of isolation and other social pressures. Seizure driving laws may prevent epileptic individuals from conducting essential daily activities including, but not limited to, grocery shopping, attending medical visits, education, childcare-related activities, and the like.

Despite advances in seizure detection research, there is still a scarcity of seizure prediction methods and devices that are inexpensive, noninvasive, and accurate. An ideal detection method would allow not only for the prediction of seizures but would also provide information in advance about the type of seizure a subject will likely experience. These needs and other needs are satisfied by the present disclosure.

### SUMMARY

In accordance with the purpose(s) of the present disclosure, as embodied and broadly described herein, the disclosure, in one aspect, relates to a sensor for predicting epileptic seizures in a subject. In an aspect, the sensor includes carbon nanotubes (CNTs) functionalized with organic molecules that can reversibly bind pre-seizure volatile organic compounds (VOCs) by means including TT-TT bonding, polar bonding, van der Waals interactions, and the like. The sensor can detect the pre-seizure VOCs from biological fluids including sweat, saliva, and breath, thus allowing the prediction of a seizure from 10 to 45 minutes before the seizure occurs, allowing a subject wearing the sensor to take appropriate actions to ensure safety and/or to alert caregivers. In an aspect, the sensor can be incorporated into a wearable device that also monitors one or more vital signs such as, for example, heart rate, body temperature, skin impedance, and/or body movements in order to predict the type of epileptic seizure that will occur. Also disclosed are methods of making the sensors and methods for predicting epileptic seizures in a subject.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1C show exemplary aspects of chip design for the disclosed devices. FIG. 1A: Each chip includes 4 source: drain channel combinations. Left: configuration with 10 source electrodes and 9 drain electrodes. Center left: configuration with 25 source electrodes and 24 drain electrodes. Center right: configuration with 50 source electrodes and 49 drain electrodes. Right: Configuration with 100 source electrodes and 99 drain electrodes. FIGS. 1B-1C: 4 of each source; drain combination are included per chip for a total of 16 sensor options, with 24 chips included (or 384 sensors included) per wafer. FIG. 1D shows an exemplary process for fabricating a disclosed chip. FIG. 1E is a photograph of an exemplary chip.

FIGS. 2A-2L show an exemplary CNT-field effect transistor (FET) fabrication process as disclosed herein. FIG. 2A: spin-coating 1.3 μm of SC1813 positive photoresist on the thermal oxide side of the wafer, FIG. 2B baking for 1 minute at 110° C., and FIG. 2C: using the MLA150, a maskless aligner, to etch the design into the applied layer of photoresist. FIG. 2D: the design is developed using MF-319 and deionized (DI) water to FIG. 2E: form the pattern etched and metallization follows to add conductive layers within the etched pattern. Metallization occurs by using the FIG. 2F e-beam evaporator method in which crucibles are filled with the desired metals, chromium (Cr) and gold (Au), and a high-intensity beam of electrons focused at the center of the crucible evaporate the metals up to FIG. 2G: the surfaces of the wafer. For the design, both the front and back sides of the wafer were applied 5 nm Cr and 55 nm Au. Cr was used to act as a connection between the silicon dioxide and gold layers because Au and SiO₂ are not very reactive. Both sides of the wafer needed metallization because the front required conductive channels from the etched design, which Au is most commonly used for, while the entire bottom of the wafer served as the back-gate of the chip, so applying a more conductive metal to serve as the electrode increases device performance. Then the excess metal must be lifted off by FIG. 2H: soaking the wafers in acetone overnight. The acetone is used to find any photoresist it can access around the design and wafer circumference to remove it and FIG. 2I: form the metal pattern. Finally, FIG. 2J: the wafer is cut into 24 chips to prepare for FIG. 2K: CNT-deposition. The FIG. 2L: final design has CNTs located across the source and drain electrodes, as shown.

FIG. 3A shows the shift of a V_{gs} (gate voltage) versus I_{d} (drain current) curve to measure horizontal change in response when no VOC is detected (lower curve) versus when the target VOC is detected (upper curve). This shift value is used to train an artificial neural network (ANN) to recognize how the target VOCs change the curve behavior. FIG. 3B shows a photomicrograph of the chip used to measure the data in FIG. 3A.

FIGS. 4A-4B show the functionalization of the CNTs with functional groups specific to each target pre-seizure VOC. Each of the 9 target VOCs have a corresponding functional group compound that is attached to the surface of the sensor. Each attachment forms a noncovalent bond, producing a reaction and thus a signal to trigger an alert for a forthcoming seizure. Attachment consists of a 4-step process, as seen in FIG. 4A. First, (3-aminopropyl)triethyoxysilane (APTES) is covalently attached to the OH-surface CNTs through a sigma bond to activate the functionalization process. APTES serves as the head of the recognition element by forming the attachment to the sensor surface through CNT bonding. Then, various acid groups whose functional groups are associated with each target VOC are converted to chlorides to allow for a stronger polar covalent bond to the APTES, releasing CH₃OH when bonded rather than H₂O. Once the OH group of each of the acids (5-phenylvaleric acid, adipic acid, and pentadecanoic acid) is replaced by CI through the application of thionyl chloride and dried DMF, the modified compounds are covalently attached to the APTES, creating a permanent connection for deposition on the sensor surface. The acyl chloride groups serve as the tails of the recognition elements, being attached to the APTES on one end, while the other end is exposed to the surrounding environment to detect the target compounds. This process allows for the acyl chlorides to selectively attach to their corresponding VOCs through noncovalent bonding, making the sensor refreshable since the VOCs can be released for a second detection following the initial alert. Menthone, L-menthyl acetate, β-cubene, (−)-β-bourbene, and valencene will utilize the acyl chloride, 5-phenylvalric chloride (5-PC), for IT-IT bonding and, once detected, the noncovalent interaction will be broken until detected again. Ethyl linalyl ether and pentadecanal will use pentadecanoyl chloride (PDC) for a van der Waals connection with the long fatty chain. Finally, 4-tert-butylcycohexyl acetate and camphor will form a noncovalent bond with the COOH functional groups in adipic coyl chloride (ACC).

FIGS. 5A-5F show the CNT functionalization process, which begins by FIG. 5A: adding APTES to the OH-CNTs suspended in pure ethanol and placing the solution under reflux for 4 hours with a condenser to prevent solvent loss. Next, FIG. 5B: the APTES-CNTs are centrifuged to a pellet the excess ethanol is decanted to eliminate unbound APTES. Then the APTES-CNT pellet is resuspended in ethanol, acetone, and water with centrifuge/empty steps in between to ensure that the APTES-CNTs are thoroughly cleaned. The CNTs are then placed in the vacuum oven to dry back to their powder form. While drying, FIG. 5C: 5-phenylvaleric acid, adipic acid, and pentadecanoic acid are placed in separate flasks under nitrogen and vacuum as their conversion to chloride is a dry reaction. Once all air and moisture have left the flasks, thionyl chloride and a small volume of dried DMF are added to the 3 flasks. The adipic acid flask also has THF added to it because in the 5-PC and PDC reactions, a 5:1 ratio of thionyl chloride:acid is used so the thionyl chloride acts as both the reactant and solvent, but for the adipic acid, a 1:1 ratio is used to prevent having an excess since the goal is to chloridate just one of the OH groups per molecule. Then, the solutions are again placed under reflux for 4-5 hours before being connected to a short path distillation apparatus under vacuum for 15-20 minutes to remove excess thionyl chloride. Once completed, the acids should now be chlorides, and FIG. 5D: the APTES-CNTs are added to 3 additional separate flasks and placed under vacuum and nitrogen before adding dried DMF and each acyl chloride (5-PC, PDC, and ACC). Then, the solution is placed back into the bath for 24 hours at 100° C. After the reaction is complete, the flask is opened to quench the unreacted acyl chlorides, and FIG. 5E: the centrifuge/empty/resuspend cycle is repeated using DI water then THF 3 times to remove excess solvent from the functionalized CNTs. Then, FIG. 5F: each of the CNTs are placed in the vacuum oven to dry and are ready for application on the CNT-FET surface.

FIG. 6 shows a sensor on the CNT-FET surface. To deposit the CNTs on the surface of the FET devices, each type of CNT is resuspended in dried DMF at a 1 mg: 1000 mL ratio with 1 hour of sonication. About 40 μL is then deposited on each chip using a pipette, and they are air-dried under a fume hood for 4 hours. Once dry, the devices are observed under an optical microscope to locate which sensors have CNTs between source and drain electrodes. The chips are tested using a probe station for electrical characterization of the functionalization, as described in FIGS. 3A-3B.

FIG. 7 shows a schematic for selective detection of pre-seizure VOCs though an artificial neural network (ANN) and gas flow chamber designed to allow the disclosed sensor array to learn the phantom mixtures of VOCs introduced.

FIG. 8 shows an exemplary overall artificial neural network (ANN) that can be used for gas flow testing. Once the 3 different channels in the model have been established (nonfunctionalized FET, functionalized FET, and ionized FET), the phantom solution matrix physical implementation begins by introducing target mixtures to the setup: VOC 1, 1-2 . . . 1-9, 2, 2-3 . . . 2-9, etc. By collecting the parameters for these arrays through up to thousands of training sets, the microcontroller develops a fingerprint for each biomarker, until it is able to distinguish each of the pre-seizure compounds through combined ANNs.

FIG. 9 shows an exploded view of an exemplary device as disclosed herein.

FIG. 10 shows an additional CNT placement confirmation method. SEM is used to look closely at where CNTs are deposited in the gate channel to ensure that circuit is closed prior to electrical characterization. 10A) SEM of channels. 10B) SEM of entire sensor.

FIG. 11A shows a Fourier Transform Infrared (FTIR) spectrum of multi-walled carbon nanotubes (MWCNTs). FIG. 11B shows an FTIR spectrum of MWCNTs+APTES. FIG. 11C shows an FTIR spectrum of OH-functionalized MWCNTs+APTES. FIG. 11D shows an expanded view of the spectrum shown in FIG. 11C.

FIG. 12A shows an FTIR spectrum of multi-walled carbon nanotubes (MWCNTs). FIG. 12B shows an FTIR spectrum of MWCNTs+APTES. FIG. 12C shows an FTIR spectrum of OH-functionalized MWCNTs+APTES. FIG. 12D shows an expanded view of the spectrum shown in FIG. 12C. The functionalization seen in FIGS. 11A-12D represent the confirmation of each bonding step of the functionalization process using FTIR to observe the addition of functional groups.

FIG. 13A shows the typical V_{gs} (gate voltage) vs I_{d} (drain current) curve for a sensor on 1 chip. The threshold voltage is at about-1-0 V, as shown in FIG. 13C, which shows the typical V_{ds} (source-drain voltage) versus I_{d} curve at different gate voltages. The typical curve starts at ˜10-10² μA and decreases down to ˜0 μA, representing the ON/OFF state of the sensor. FIG. 13B shows a laser-illuminated image of the sensor with the red dots illustrating the CNTs in the gate channel. FIG. 13D shows a zoomed-in image using SEM of the sensor above. The gate channel is made from small CNT networks which allow for higher sensitivity of each sensor. By identifying the threshold voltage at about-1 to 0 V, the shift of the curve up and to the right can be measured, as seen in FIG. 14.

FIG. 14 shows VOC Detection Using CNT-FET. Different target VOCs were introduced to the sensor and caused a shift upward and slightly to the right for each new VOC introduced from the control curve (bottom). The sensor is able to discriminate different signals for each target VOC. These signals are used to assign VOC fingerprints to the microcontroller for selectivity.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

As our understanding of the olfactory system has expanded alongside our advancements in odor detection, ample research has been conducted in the past decade about what individual odors are associated with various diseases. Animals and “electronic noses” are the key indicators of establishing these odor-disease relationships and have opened the doors to early detection of many disorders, including epileptic seizures. Many publications support the notion that seizure alert dogs are able to detect seizures 10-45 minutes prior to their occurrence with up to 95% accuracy; however, there has yet to be a device that uses the pre-seizure odor as its sensing mechanism. The work of Maa et al. presents the first study to identify a specific pre-seizure biomarker characterized by odor combinations of VOCs present in a control group of 60 patients over the course of 2 years by collecting samples from saliva, sweat, and breath of various epileptic individuals during the immediate ictal period (start of the seizure). This profile includes menthone, L-menthyl acetate, ethyl linalyl ether, camphor, pentadecanal, valencene, (−)-β-bourbene, β-cubebene, and 4-tert-butylcyclohexyl acetate.

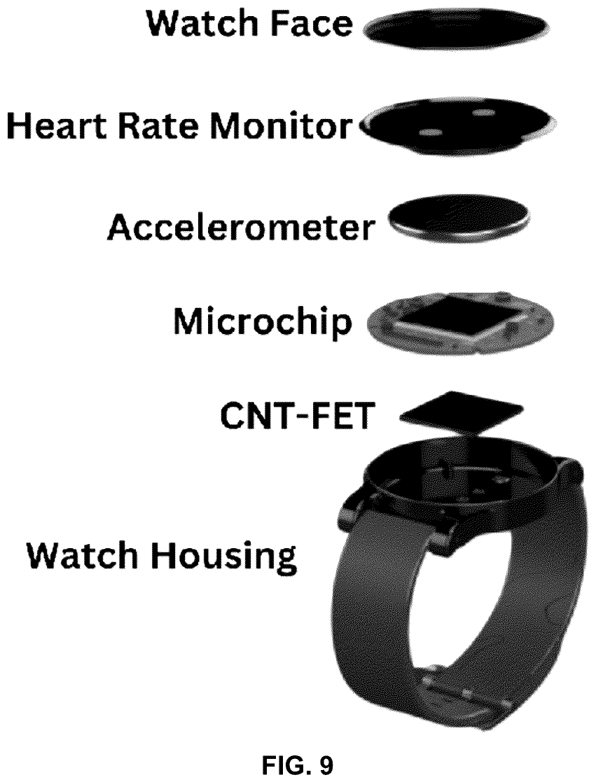

Disclosed herein are a method and a device for detecting these odor emissions, the method making use of a wearable sensor and not requiring the use of expensive and rare trained seizure-detection dogs. In one aspect, the sensor can be part of a watch (FIG. 9) or can be designed to be worn on the wrist. The disclosed method and device can detect at least 9 pre-seizure VOCs and/or other VOCs associated with epileptic seizures at the ppb level and can differentiate pre-seizure VOCs from the other 532 known VOCs that the human body releases including those associated with other disorders and diseases. The disclosed method and device are also able to differentiate VOCs emitted by a wearer of the sensor from those present in the atmosphere.

Disclosed herein are a new method and system for seizure detection that incorporates chemoreception and has a low rate of false positive reports. In one aspect, current wearable pre-seizure detection devices have high false positivity rates due to their triggering mechanisms. These alternatives use various changes in physiological parameters such as heart rate and body temperature, motion, skin impedance, and vagus nerve response. However, such physiological parameters are also associated with a multitude of non-seizure related activities including daily exercise, stressful situations, and even sleep. In another aspect, current devices only provide an alert seconds to a few minutes prior to the seizure occurring, which can be insufficient for proper precautions to be taken. In another aspect, the leading surgically implanted seizure detection device, Vagus Nerve Stimulation (VNS) suffers from the same drawbacks as well as requiring an invasive implantation procedure and due to the electrical pulse is sent to the epileptic individual's brain to suppress the episodes for individual with focal (partial) seizures, even when a false alert is signaled.

In one aspect, before someone has a seizure, their skin, saliva, and breath release 9 volatile organic compounds (VOCs), which can be sensed by seizure alert dogs from about 10-45 minutes prior to an episode. In another aspect, the disclosed device incorporates a sensor that accurately detect seizures by a noninvasive means. In an aspect, nonwearable CNT-FET devices created for detection of liquid medium which incorporate other types of recognition elements, such as antibodies and enzymes, are known. However, no existing wearable gas sensor that uses CNT-FET principles and incorporates the method of selective detection disclosed herein has been described. In some aspects, the devices can incorporate a photoionization detector (PID) for a more accurate selective reading of the target gas molecules, which will also allow the determination of concentrations of VOCs released.

In an aspect, the fabricated VOC sensor arrays and PID will be used to denote seizure presence, and vital sensors will be added to the device to monitor what heart rate, body temperature, skin impedance, and movements are associated with various types of seizures that an individual may have.

In one aspect, highest and lowest ictal heart rate can be determined, where the ictal stage of a seizure is defined as the middle stage of a seizure. In some aspects, the heart rate can be less than 60 beats per minute and may be defined as bradycardia. In other aspects, the heart rate can exceed 100 beats per minute and may be defined as tachycardia. In either of these aspects, a change in heart rate such as, for example, bradycardia or tachycardia may be indicative of a particular type of seizure in an individual subject.

In another aspect, skin impedance can be measured or assessed in several ways. In one aspect, galvanic skin response (GSR) is defined as a change in the electrical properties of the skin. In a further aspect, this response can appear as an increase in the electrical conductance of the skin (i.e., a decrease in resistance) across the palms of the hands or soles of the feet. In another aspect, electrical impedance tomography (EIT) is an imaging method in which the internal impedance of the subject can be imaged using rings of external electrodes. In either of these aspects, feedback from a device for monitoring GSR or performing EIT can be useful in classifying seizure type.

In still another aspect, elevation of body temperature above 37.8° C. (100° F.) may be indicative of a particular seizure type in an individual subject and can be measured by a sensor incorporated into the disclosed devices. In yet another aspect, an accelerometer can be incorporated into the disclosed devices and signals from the accelerometer may further be useful in classifying seizure type.

### Sensors

In one aspect, disclosed herein is a sensor for predicting epileptic seizures in a subject, the sensor including a chip with a layer of carbon nanotubes (CNTs), wherein the CNTs are functionalized to include a reactive species that is chemically bonded to at least one recognition element, and wherein the at least one recognition element non-covalently binds with one or more pre-seizure VOCs. In a further aspect, the pre-seizure VOCs are emitted by the subject in saliva, sweat, breath, or any combination thereof. In some aspects, the sensor can be worn by the subject on or near a body surface that emits saliva, sweat, or breath, such as, for example, the wrist, underarm, arm, neck, or another skin surface. In one aspect, the sensor is, or is a component of, a smart watch, armband, necklace, or skin patch.

In one aspect, the CNTs can be functionalized with (3-aminopropyl) triethyoxysilane (APTES). In another aspect, the chip includes a discontinuous SiO₂ having exposed areas of silicon and areas of SiO₂, and further in this aspect, at least one metal can be in contact with the at least a portion of the exposed areas of silicon, wherein a “portion” is defined as an area greater than zero percent of the exposed areas of silicon but less than the entirety of the exposed areas of silicon. In some aspects, the CNTs do not form a continuous layer on the chip but are deposited such that they contact at least a portion of the exposed areas not in contact with the metal, such that the CNTs form connections between source and drain electrodes formed from the metal. In an aspect, the metal can be Cr, Au, or any combination thereof. In one aspect, when the at least one metal includes Cr, the Cr can have a thickness of about 5 nm. In another aspect, when the metal includes Au, the Au can have a thickness of about 55 nm. In a further aspect, the combined thickness of the Cr and Au is equal to 60 nm, or less than 100 nm, so as to not penetrate completely through the thermal oxide layer to the back gate (silicon). In some aspects, the metal can be both Cr and Au provided in separate layers. Further in this aspect, and without wishing to be bound by theory, the Au may provide a better electrical contact than the Cr, but is unreactive with the silicon of the chip, so a Cr layer can be deposited first, followed by an Au layer. In any of these aspects, the CNTs are deposited on the at least one exposed areas of SiO₂. In another aspect, the CNT deposition connects the source and drain electrodes from the channels on the surface of the sensor chip. In some aspects, and without wishing to be bound by theory, Au is too reactive to be used alone in the metal layer, so nonreactive Cr can be used in conjunction with the Au. In some aspects, and without wishing to be bound by theory, Cr is too conductive to be used as an electrode material alone in the disclosed sensor, so Cr can instead be used in conjunction with Au.

In any of these aspects, the one or more pre-seizure VOCs can be selected from menthone, L-menthyl acetate, valencene, β-cubebene, (−)-β-bourbene, 4-tert-butylcyclohexyl acetate, camphor, pentadecanal, ethyl linalyl ether, or any combination thereof. In another aspect, the at least one recognition element can be 5-phenylvaleric acid, adipic acid, pentadecanoic acid, reacted with thionyl chloride to produce 5-phenylvaleric chloride, adipic coyl chloride, pentadecenoyl chloride, or any combination thereof. In some aspects, the chip includes all three recognition elements. In an aspect, the CNTs contain hydroxyl groups which are reacted with (3-aminopropyl) triethyoxysilane (APTES), and the APTES is then reacted with 5-phenylvaleric chloride, adipic coyl chloride, pentadecenoyl chloride, or any combination thereof, in order to link the recognition elements to the disclosed chips. In one aspect, the CNTs are provided with already existing hydroxyl groups. In an alternative aspect, the CNTs can be functionalized with OH groups using KMnO₄. An exemplary procedure for adding OH groups to CNTs is provided in the Examples.

In a further aspect, when the at least one recognition element is 5-phenylvaleric acid (5-phenylvaleric chloride), the pre-seizure VOCs interact with the recognition element via IT-IT bonding with an aromatic ring in the recognition element. Further in this aspect, the one or more pre-seizure VOCs can be menthone, L-menthyl acetate, valencene, β-cubebene, (−)-β-bourbene, or any combination thereof. In another aspect, when the at least one recognition element is adipic acid (adipic coyl chloride), the pre-seizure VOCs interact with the recognition element via polar bonding with a carboxylic acid group in the adipic acid. Further in this aspect, the one or more pre-seizure VOCs can be 4-tert-butylcyclohexyl acetate, camphor, (−)-β-bourbene, or any combination thereof. In still another aspect, when the at least one recognition element is pentadecanoic acid (pentadecenoyl chloride), the pre-seizure VOCs can interact with the recognition element via van der Waals interactions with a long fatty chain in the recognition element. Further in this aspect, the one or more pre-seizure VOCs can be pentadecanal, ethyl linalyl ether, or any combination thereof.

In still another aspect, the CNTs can be single walled carbon nanotubes (SWCNTs), multi walled carbon nanotubes (MWCNTs), or any combination thereof. In a preferred embodiment, SWCNTs are used. Without wishing to be bound by theory, SWCNTs are semi-conductive and a semiconductive nanomaterial offers better control over the electrical properties of the sensor. In some aspects, the CNTs have an average length of about 100 nm to about 30 μm, or about 5 μm, and an average diameter of from about 1 to about 4 nm, or of about 1, 2, 3, or 4 nm, or a combination of any of the foregoing values, or a range encompassing any of the foregoing values. In some aspects, and without wishing to be bound by theory, longer CNTs may be better able to stretch across source and drain electrodes on the sensor.

In one aspect, a semiconductor is preferable for a FET device. Conductive materials allow electron flow too easily, but semiconductive materials allow for appropriate transitions. The overlapping chirality of walls in MWCNTs can create a nonzero band gap causing conductivity, leading the device to always be on, while semiconductors can allow the device to switch between on and off.

When a voltage is applied to the gate, it creates an electric field that can either attract or repel the electrons in the carbon nanotube channel. In a “semiconducting” carbon nanotube, which is often used in CNT-FETs, the electrons are confined to energy levels within the tube, and the electric field can change the energy levels of the electrons.

When the gate voltage is low, the carbon nanotube is in a “depletion” state, meaning that there is a barrier to the flow of electrons between the source and drain terminals. When the gate voltage is increased, the barrier becomes smaller, and the electrons can begin to flow through the channel. At a certain gate voltage, called the “threshold voltage,” the barrier disappears completely, and the CNT-FET enters an “on” state, allowing current to flow freely between the source and drain.

The current that flows through the carbon nanotube channel is carried by the electrons that move through the energy levels within the tube. The resistance of the carbon nanotube channel can be very low, allowing for high current densities and fast switching speeds in CNT-FETs.

In one aspect, and without wishing to be bound by theory, when a VOC binds to the surface of the carbon nanotube, it can introduce charge carriers or modify the electronic structure of the nanotube. This affects the conductivity of the nanotube channel and the overall performance of the transistor. VOC molecules can donate or accept electrons from the nanotube, leading to a change in the overall charge carrier concentration in the channel. This can influence the conductivity of the nanotube, leading to a shift in the I_{d}-V_{gs} curve. The binding of VOCs can act as a doping agent, altering the carrier concentration in the nanotube. Doping can introduce extra charge carriers (n-type or p-type) and affect the threshold voltage, causing a shift. VOCs can induce changes in the electronic structure of the nanotube, affecting its band structure or energy levels. This can result in a modification of the transistor's threshold voltage and conductance. The binding of VOCs can alter the surface potential of the nanotube, influencing the electrostatics within the transistor and shifting the I_{d}-V_{gs} curve.

### Methods for Making the Sensors

In another aspect, disclosed herein is a method for making the disclosed sensors, the method including at least the steps of:
(a) providing a silicon wafer;
(b) oxidizing a SiO₂ layer on the silicon wafer;
(c) performing a lithography step to etch a lithography pattern in the SiO₂ layer;
(d) evaporating one or more metals on the exposed lithography pattern to create a plurality of source electrodes and drain electrodes on the wafer; and
(e) depositing functionalized CNTs on the chip.

In one aspect, the SiO₂ layer is from about 50 nm to about 100 nm thick, or is about 50, 60, 70, 80, 90, or about 100 nm thick, or a combination of any of the foregoing values, or a range encompassing any of the foregoing values. In some aspects, the chip can be p-doped. In a further aspect, the method can be performed on a wafer that includes two or more chips.

In another aspect, the method further includes functionalizing the CNTs with at least one recognition element prior to performing the method. In an aspect, functionalizing the CNTs includes at least the steps of:
(a) covalently attaching (3-aminopropyl) triethoxysilane (APTES) to OH groups on a surface of the CNTs;
(b) reacting at least one recognition element precursor with thionyl chloride to product an acyl chloride; and
(c) reacting the acyl chloride with the APTES.

In a further aspect, the at least one recognition element precursor includes 5-phenylvaleric acid, adipic acid, pentadecanoic acid and where the acyl chlorides are selected from 5-phenylvaleric chloride, adipic coyl chloride, and pentadecenoyl chloride, respectively.

### Methods for Predicting Seizures

Also disclosed herein is a method for predicting an epileptic seizure in a subject, the method including using the disclosed sensors to sense emission by the subject of one or more pre-seizure VOCs. In a further aspect, the epileptic seizure can be predicted from about 10 minutes to about 45 minutes before occurring, or about 10, 15, 20, 25, 30, 35, 40, or about 45 minutes before occurring, or a combination of any of the foregoing values, or a range encompassing any of the foregoing values.

In another aspect, the method further includes collecting at least one vital sign from the subject. Further in this aspect, the at least one vital sign can be heart rate, body temperature, skin impedance, body movements, or any combination thereof. In another aspect, the at least one vital sign can be correlated to epileptic seizure type. Further in this aspect, the epileptic seizure type can be an absence seizure, a tonic-clonic seizure, a simple focal seizure, a complex focal seizure, a secondary generalized seizure, or any combination thereof. In some aspects, the sensor and/or a device including the sensor can be trained by an artificial neural network to correlate VOCs and/or vital signs to seizure type for each individual subject.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a volatile organic compound,” “a vital sign,” or “an electrode metal,” include, but are not limited to, mixtures or combinations of two or more such volatile organic compounds, vital signs, or electrode metals, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of functionalized carbon nanotubes refers to an amount that is sufficient to achieve the desired effect modulated by the functionalized carbon nanotubes, e.g. achieving the desired level of reactivity with volatile organic compound recognition elements in order to construct a functional sensor. The specific level in terms of wt % in a composition required as an effective amount will depend upon a variety of factors including the amount and type of carbon nanotubes, conductive or semi-conductive quality of the carbon nanotubes, number of sensors being constructed, and similar factors.

As used herein, the terms “optional” or “optionally” means that the subsequently described event or circumstance can or cannot occur, and that the description includes instances where said event or circumstance occurs and instances where it does not.

A “volatile organic compound” or VOC is a compound with a high vapor pressure and low water solubility. VOCs can be found in the environment and the human body, among other places. VOCs are endogenous products of physiological and/or metabolic body processes or can be exogenous products of the external environment (e.g. related to eating and drinking). VOC may be emitted from the skin, saliva, urine, feces, and in the breath as well as being found in other bodily fluids and secretions. In some aspects, a different VOC profile may occur in a diseased metabolic state relative to the VOC profile of a healthy subject.

A “field effect transistor” (FET) as used herein is a triode semiconductor device that achieves current control by changing the internal electric field of the semiconductor. An FET includes a source and a drain with a gate being the semiconductor channel layer between source and drain. The dielectric layer of an FET can control the current in the channel layer when gate voltage is applied. In one aspect, when a compound of interest interacts with an immobilized recognition element, a change of channel surface charge transfer is induced, resulting in a change of channel conductance and successful detection of target molecules. FETs have high sensitivity, can easily be miniaturized, and offer real-time detection. A “CNT-FET” is a FET that includes carbon nanotubes. A CNT-FET works similarly to a FET but the channel is made up of one or a few CNTs. In a CNT-FET, the carbon nanotube acts as the channel between the source and drain terminals, and the gate electrode controls the flow of current through the channel. When a voltage is applied to the gate, it creates an electric field that changes the electronic properties of the CNT channel. When the gate voltage is high enough, it creates a channel in the CNT that allows current to flow, turning the CNT-FET on.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered aspects, which should not be confused with the claims.

Aspect 1. A sensor for predicting epileptic seizures in a subject, the sensor comprising a chip comprising a layer of carbon nanotubes (CNTs),
wherein the CNTs are functionalized to comprise a reactive species that is chemically bonded to at least one recognition element;
wherein the at least one recognition element non-covalently binds with one or more pre-seizure volatile organic compounds (VOCs).

Aspect 2. The sensor of aspect 1, wherein the pre-seizure VOCs are emitted by the subject in saliva, sweat, breath, or any combination thereof.

Aspect 3. The sensor of aspect 1 or 2, wherein the sensor is worn by the subject on or near a body surface that emits saliva, sweat, breath, or any combination thereof.

Aspect 4. The sensor of aspect 3, wherein the body surface comprises the wrist, underarm, arm, neck, or another skin surface.

Aspect 5. The sensor of aspect 3 or 4, wherein the sensor is, or is a component of, a smart watch, armband, necklace, or skin patch.

Aspect 6. The sensor of any one of aspects 1-5, wherein the CNTs are functionalized with (3-aminopropyl) triethyoxysilane (APTES).

Aspect 7. The sensor of any one of aspects 1-6, wherein the chip comprises a discontinuous SiO₂ layer comprising exposed areas of silicon and areas of SiO₂.

Aspect 8. The sensor of aspect 7, further comprising at least one metal in contact with at least a portion of the exposed areas of silicon.

Aspect 9. The sensor of aspect 8, wherein the at least one metal has a thickness of between about 50 nm and about 100 nm.

Aspect 10. The sensor of aspect 8 or 9, wherein the at least one metal comprises Cr, Au, or any combination thereof.

Aspect 11. The sensor of aspect 10, wherein the Cr has a thickness of about 5 nm.

Aspect 12. The sensor of aspect 10, wherein the Au has a thickness of about 55 nm.

Aspect 13. The sensor of any one of aspects 1-12, wherein the CNTs are deposited on the exposed areas of SiO₂.

Aspect 14. The sensor of any one of aspects 1-13, wherein the one or more pre-seizure VOCs comprise menthone, L-menthyl acetate, valencene, β-cubebene, (−)-3-bourbene, 4-tert-butylcyclohexyl acetate, camphor, pentadecanal, ethyl linalyl ether, or any combination thereof.

Aspect 15. The sensor of any one of aspects 1-14, wherein the at least one recognition element comprises 5-phenylvaleric chloride, adipic coyl chloride, pentadecanoyl chloride, or any combination thereof.

Aspect 16. The sensor of aspect 15, wherein the at least one recognition element is 5-phenylvaleric chloride and interacts with the one or more pre-seizure VOCs via TT-TT bonding with an aromatic ring in the 5-phenylvaleric chloride.

Aspect 17. The sensor of aspect 16, wherein the one or more pre-seizure VOCs comprise menthone, L-menthyl acetate, valencene, β-cubebene, (−)-β-bourbene, or any combination thereof.

Aspect 18. The sensor of aspect 15, wherein the at least one recognition element is adipic coyl chloride and interacts with the one or more pre-seizure VOCs via polar bonding with a carboxylic acid group in the adipic coyl chloride.

Aspect 19. The sensor of aspect 18, wherein the one or more pre-seizure VOCs comprise 4-tert-butylcyclohexyl acetate, camphor, (−)-β-bourbene, or any combination thereof.

Aspect 20. The sensor of aspect 15, wherein the at least one recognition element is pentadecanoyl chloride and interacts with the one or more pre-seizure VOCs via van der Waals interactions with a long fatty chain in the pentadecanoyl chloride.

Aspect 21. The sensor of aspect 20 wherein the one or more pre-seizure VOCs comprise pentadecanal, ethyl linalyl ether, or any combination thereof.

Aspect 22. The sensor of any one of aspects 1-21, wherein the carbon nanotubes comprise single walled carbon nanotubes (SWCNTs), multi walled carbon nanotubes (MWCNTs), or any combination thereof.

Aspect 23. The sensor of aspect 22, wherein the carbon nanotubes are SWCNTs.

Aspect 24. The sensor of aspect 22 or 23, wherein the carbon nanotubes have an average length of from about 100 nm to about 30 μm.

Aspect 25. The sensor of aspect 24, wherein the carbon nanotubes have an average length of about 5 μm.

Aspect 26. The sensor of any one of aspects 22-25, wherein the carbon nanotubes have an average diameter of from about 1 to about 4 nm.

Aspect 27. A method for making the sensor of any one of aspects 1-26, the method comprising:
(a) providing a silicon wafer;
(b) oxidizing a SiO₂ layer on the silicon wafer;
(c) performing a lithography step to etch a lithography pattern in the SiO₂ layer;
(d) evaporating one or more metals on the exposed lithography pattern to create a plurality of source electrodes and drain electrodes on the wafer; and
(e) depositing functionalized CNTs on the chip.

Aspect 28. The method of aspect 27, wherein the outer SiO₂ layer is from about 50 nm to about 100 nm thick.

Aspect 29. The method of aspect 27 or 28, wherein the chip is p-doped.

Aspect 30. The method of any one of aspects 27-29, wherein the method is performed on a wafer comprising two or more chips.

Aspect 31. The method of any one of aspects 27-30, wherein the one or more metals comprise Cr, Au, or any combination thereof.

Aspect 32. The method of any one of aspects 27-31, wherein the one or more metals collectively comprise a thickness of less than or equal to 100 nm.

Aspect 33. The method of aspect 31 or 32, wherein the Cr has a thickness of about 5 nm.

Aspect 34. The method of aspect 32 or 32, wherein the Au has a thickness of about 55 nm.

Aspect 35. The method of any one of aspects 27-34, wherein the functionalized CNTs comprise single walled carbon nanotubes (SWCNTs), multi walled carbon nanotubes (MWCNTs), or any combination thereof.

Aspect 36. The method of aspect 35, wherein the functionalized carbon nanotubes are SWCNTs.

Aspect 37. The method of aspect 35 or 36, wherein the carbon nanotubes have an average length of from about 100 nm to about 30 μm.

Aspect 38. The method of aspect 37, wherein the functionalized CNTs have an average length of about 5 μm.

Aspect 39. The method of any one of aspects 35-38, wherein the functionalized CNTs have an average diameter of from about 1 nm to about 4 nm.

Aspect 40. The method of aspect any one of aspects 27-39, further comprising functionalizing the CNTs with at least one recognition element prior to performing the method.

Aspect 41. The method of aspect 40, wherein functionalizing the CNTs comprises:
(a) covalently attaching (3-aminopropyl) triethoxysilane (APTES) to OH groups on a surface of the CNTs;
(b) reacting at least one recognition element precursor with thionyl chloride to product an acyl chloride; and
(c) reacting the acyl chloride with the APTES.

Aspect 42. The method of aspect 41, wherein the at least one recognition element precursor comprises 5-phenylvaleric acid, adipic acid, pentadecanoic acid, or any combination thereof.

Aspect 43. A method of predicting an epileptic seizure in a subject, the method comprising using the sensor of any one of aspects 1-26 to sense emission by the subject of one or more pre-seizure VOCs.

Aspect 44. The method of aspect 43, wherein the epileptic seizure is predicted from about 10 minutes to about 45 minutes prior to occurring.

Aspect 45. The method of aspect 43 or 44, wherein the method further comprises collecting at least one vital sign from the subject.

Aspect 46. The method of aspect 45, wherein the at least one vital sign comprises heart rate, body temperature, skin impedance, body movements, or any combination thereof.

Aspect 47. The method of aspect 45 or 46, wherein the at least one vital sign is correlated to epileptic seizure type.

Aspect 48. The method of aspect 47, wherein the epileptic seizure type comprises an absence seizure, a tonic-clonic seizure, a simple focal seizure, a complex focal seizure, a secondary generalized seizure, or any combination thereof.

### Examples

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Fabrication of Carbon Nanotube Field Effect Transistors (CNT-FETs) for Platform and Nanomaterial of Pre-Seizure Detection Sensor

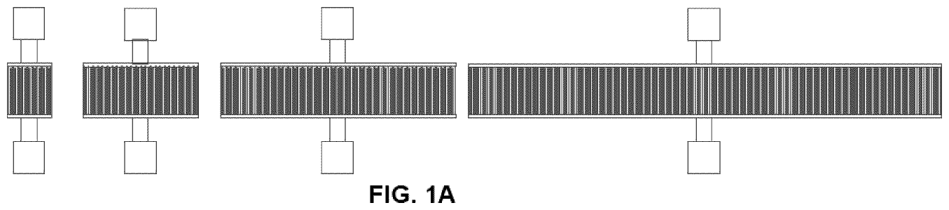

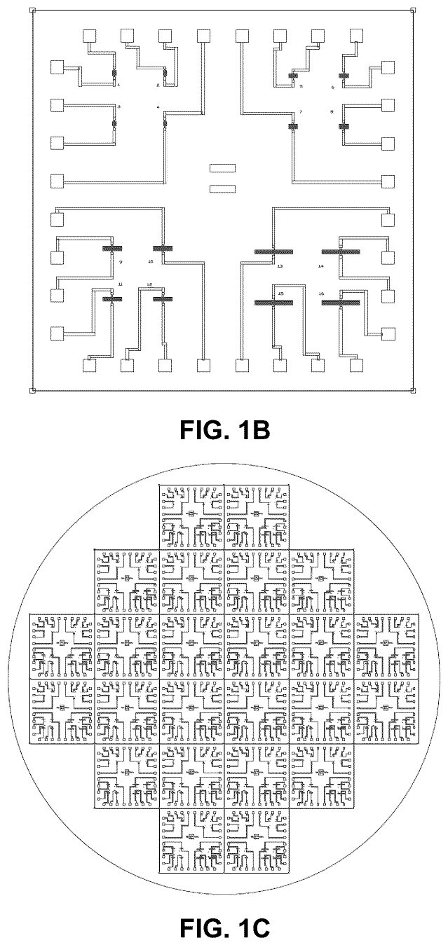

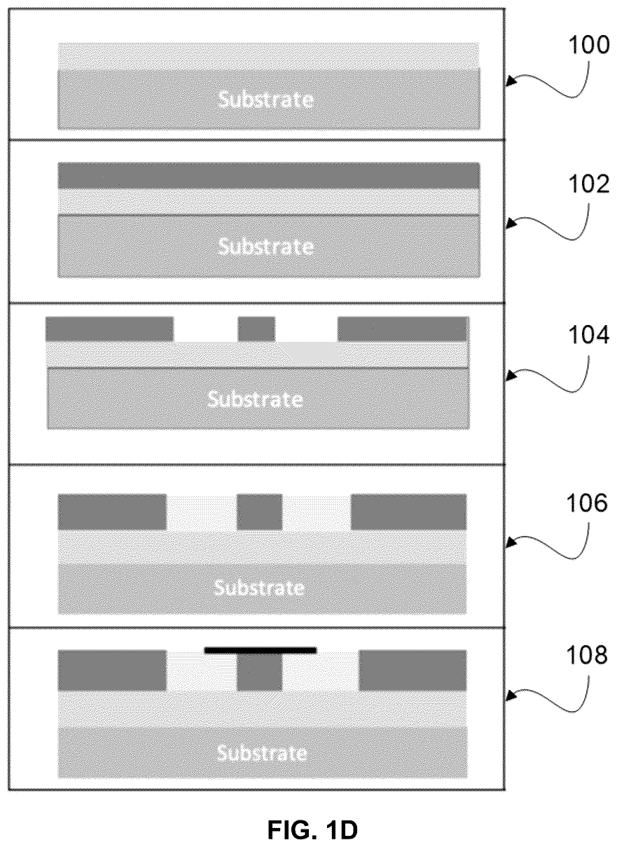

FIG. 1D shows an exemplary fabrication process, which is described in more detail below. A silicon dioxide layer is deposited on a substrate in step 100. A photoresist is applied to the top of the silicon dioxide layer in step 102. One or more openings in the photoresist are generated by etching in step 104. A metal layer, such as one consisting of Cr and Au, is deposited, with liftoff as needed, in step 106. Carbon nanotubes are drop-casted on top of the metal layer in step 108.

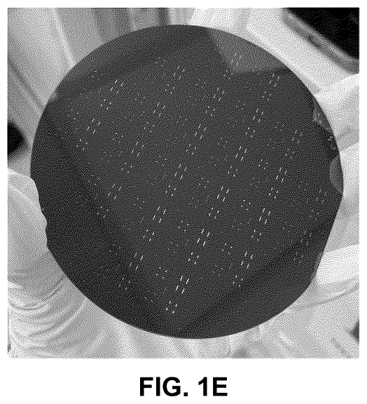

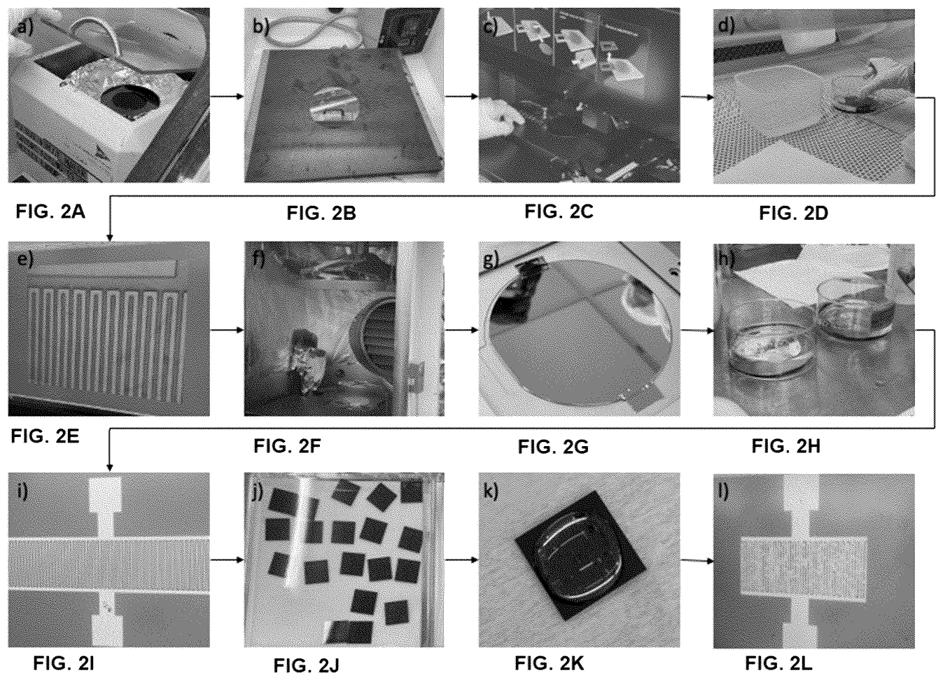

The fabrication of each chip begins with the FET CAD design to be printed on each 4-inch unpolished p-doped silicon wafer through the determination of channel layout, calculation of channel quantities and dimensions, number of chips per wafers, as well as electrode size and location. The design was created as an interlocked finger pattern of varying source to drain electrode channel ratios (see FIG. 1A) so that functionalized CNTs could be deposited between the electrodes to form the gate channel for VOC detection. Once the design is complete, its orientation on the wafer for most efficient chip distribution is determined; the final layout can be seen in FIGS. 1B-1C, with a photograph of a fabricated wafer shown in FIG. 1E. It was determined that 4 source: drain channel combinations were to be tested with 4 of each combination type per chip for a total of 16 sensor options per chip, 24 chips per wafers, and 384 sensors per wafer. Next, the thickness of the thermal oxide must be researched to conclude the appropriate silicon dioxide layer that balances dielectric protection from leakage current with the back gate's device functionality. The most common thicknesses known in the art are between 50-100 nm for back-gate FET devices. Dry oxidation is the method used to apply about a 75 nm SiO₂ layer and parameters to achieve this thickness, such as temperature and time, were calculated using the oxide growth calculator. The subsequent fabrication steps of the process flow entail removing thermal oxide from one side of the wafer through the reactive ion etching method (RIE) to expose the silicon substrate for back-gate utilization, then lithography, metallization, and CNT-deposition, as shown in FIGS. 2A-2L.

FIGS. 2A-2L show an exemplary CNT-FET fabrication process as disclosed herein. FIG. 2A: spin-coating 1.3 μm of SC1813 positive photoresist on the thermal oxide side of the wafer, FIG. 2B baking for 1 minute at 110° C., and FIG. 2C: using the MLA150, a maskless aligner, to etch the design into the applied layer of photoresist. FIG. 2D: the design is developed using MF-319 and DI water to FIG. 2E: form the pattern etched and metallization follows to add conductive layers within the etched pattern. Metallization occurs by using the FIG. 2F e-beam evaporator method in which crucibles are filled with the desired metals, chromium (Cr) and gold (Au), and a high-intensity beam of electrons focused at the center of the crucible evaporate the metals up to FIG. 2G: the surfaces of the wafer. For the design, both the front and back sides of the wafer were applied 5 nm Cr and 55 nm Au. Cr was used to act as a connection between the silicon dioxide and gold layers because Au and SiO₂ are not very reactive. Both sides of the wafer needed metallization because the front required conductive channels from the etched design, which Au is most commonly used for, while the entire bottom of the wafer served as the back-gate of the chip, so applying a more conductive metal to serve as the electrode increases device performance. Then the excess metal must be lifted off by FIG. 2H: soaking the wafers in acetone overnight. The acetone is used to find any photoresist it can access around the design and wafer circumference to remove it and FIG. 2I: form the metal pattern. Finally, FIG. 2J: the wafer is cut into 24 chips to prepare for FIG. 2K: CNT-deposition. The FIG. 2L: final design has CNTs located across the source and drain electrodes, as shown. For the final CNT deposition step, functionalized single-wall CNTs (SWCNTs) of zigzag chirality are deposited on the chip surface. This application begins by making a suspense 1 mg of functionalized SWCNTs with diameters between 1-4 nm and lengths of 10 μm in 1 mL of DI water, for each chip using a probe sonicator for 1 hour. Using a pipette tip, 1 drop of the functionalized CNT solution is deposited on the chip surface, then each chip is placed in a vacuum oven for 2 hours until dry. After drying, the CNT placement is checked with an optical microscope to ensure that at least one of the CNT networks connects one of the source and drain channels. Only one network needs to be in contact on 1/16 sensors per chip for the device to function. An exemplary chip according to the present disclosure is shown in FIG. 6.

Additional process steps and parameters are described in detail below.

A dielectric layer of SiO₂ between 50 and 100 nm thick is first created on a silicon wafer. Parameters given herein are for a 50 nm thick layer and may need to be adjusted for thicker dielectric layers. A Mini Tystar Tube system is used in oxidation mode for from 10 to 25 wafers at once. When the oxidation program is complete, a Nanospec Microscope is used to verify thickness of the oxide layer by measuring reflected light waves. Reactive ion etching is used to remove SiO₂ from one side of the wafer. Removal is verified by appearance, with the SiO₂ layer having a lighter color and the silicon layer having a darker color.

Photoresist is spin coated onto the oxide layer. When photoresist deposition is complete, the wafer is baked on a hot plate for 60 s at 110° C. The layer is approximately 1.3 μm thick.

A Heidelberg Maskless Aligner (MLA 150) is used to etch a pattern in the photoresist. Exposure dose is 170 mJ/cm² for a 375 nm laser and 200 mJ/cm² for a 405 nm laser. The photoresist is developed to expose the pattern provided on the mask. A beaker is rinsed with DI water and filled with enough MF-319 Developer (Kayaku Advanced Materials, Inc.) to cover the wafer. A second beaker is rinsed and filled with DI water. The wafer is placed in the developer beaker for 30-35 s with gentle shaking. When development is completed, the wafer is placed in the DI water beaker for 5 s and rinsed with a gentle flow of DI water. The sample is dried with nitrogen. Wafers are checked microscopically to locate shorts, debris, and/or misprints as needed.

Metal layers are deposited onto the pattern on the front of the wafer using e-beam evaporation. A total chromium thickness of 5 nm is deposited at a rate of 1 [symbol] /s, followed by a 55 nm thickness of gold being deposited at a rate of 2 [symbol] /s.

Wafers are placed in a beaker with acetone and left from 4 h to overnight. The wafer is rinsed with a mixture of isopropanol and DI water, placed in a beaker of acetone, and dried with N₂. The wafer is checked microscopically to ensure excess metal is lifted. If not, the wafer is sonicated for 2 minutes and the isopropanol/DI water and acetone rinses are repeated.

A protective layer of photoresist (about 1.3 μm thick) is spin coated onto the front of the wafer. When photoresist deposition is complete, the wafer is baked on a hot plate for 60 s at 110° C.

Metal layers are deposited onto the back of the wafer using e-beam evaporation. A total chromium thickness of 5 nm is deposited at a rate of 1 [symbol] /s, followed by a 55 nm thickness of gold being deposited at a rate of 2 [symbol] /s.

The wafer is diced into chips using a DAD3361 Disco Automatic Dicing Saw. A layer of adhesive is rolled out and the protective coating is removed. A wafer holder is placed on the plastic and excess plastic is trimmed away. The wafer is placed in the center of the holder and the cutting program is activated. Chips are removed from the adhesive layer.

Photoresist is lifted off after cutting in preparation of CNT deposition. Chips are covered with acetone and soaked for 2 minutes, then transferred to soak in isopropyl alcohol for less than one minute. Samples are dried with N₂.

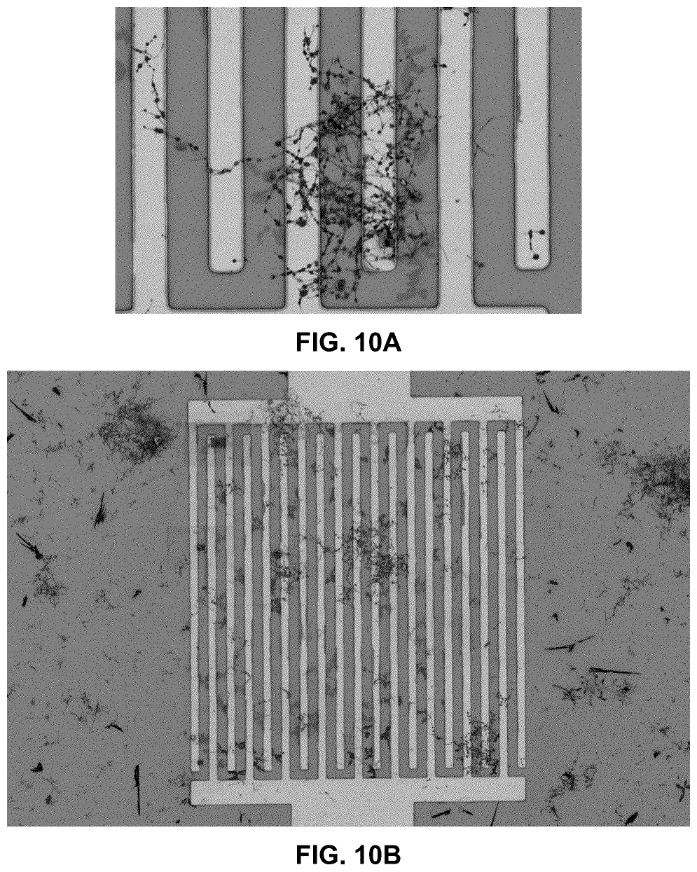

Chips are rinsed with acetone for about 30 s, then methanol, isopropanol, and DI water, successively. Chips are dried with N₂. Electron micrographs of CNTs deposited on chips are shown in FIGS. 10A-10B.

When fabrication is complete, the chips will include metal fingers composed of source and drain electrodes in an interlocked finger design. The fingers are approximately 200 μm in length and 5 μm in width and are separated by a gap of about 3 μm. Modified SWCNTs will be drop-cast between the electrodes using a polymer well/channel. A 500 μm p-doped bulk silicon layer that has a thermal oxide layer applied will serve as back gate electrode.

Drain current varies based on the thickness of the thermal oxide, with drain current being about 14 μA for a 300 nm SiO₂ thickness, and drain current being about 34 μA when the SiO₂ thickness is about 50 nm, for the same carbon nanotube length (about 1000 nm). Although 50 nm thickness for the thermal oxide layer results in a slight loss in leakage current reduction, the switching speed/gate capacitance is increased.

Following preparation of the chips, functionalized CNTs are deposited. (Functionalization of CNTs is described in Example 2.) CNTs and water in a 1:2 ratio are placed in a beaker and sonicated. Ice can be added to prevent temperature increase. 1 mg of CNTs are deposited on each chip using a pipette with a pipette and the chip is placed in a vacuum oven to dry for 3-4 hours at 70° C. A microscope can be used to check CNT placement between channels. In some experiments, more water is used per 1 mg of CNTs to fine-tune number of CNTs applied to the chip.

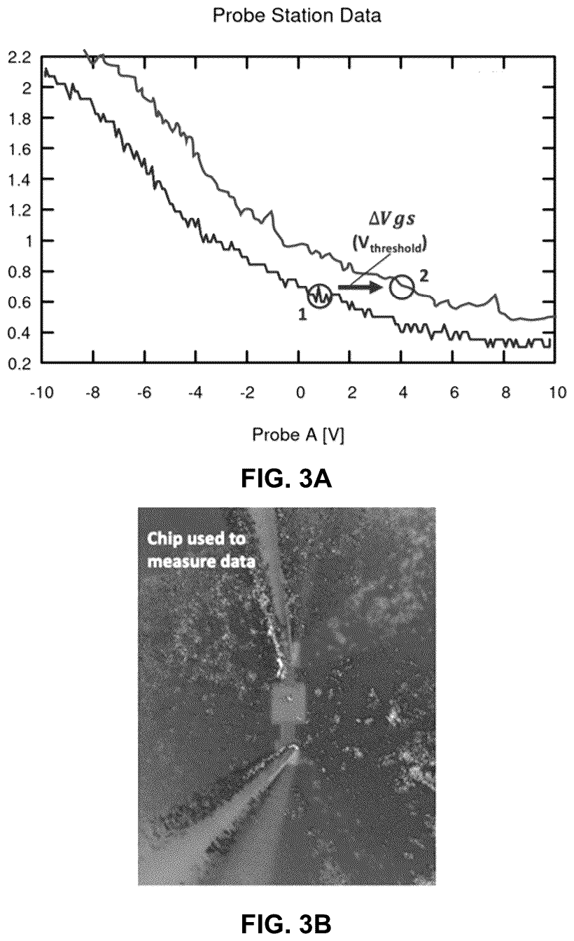

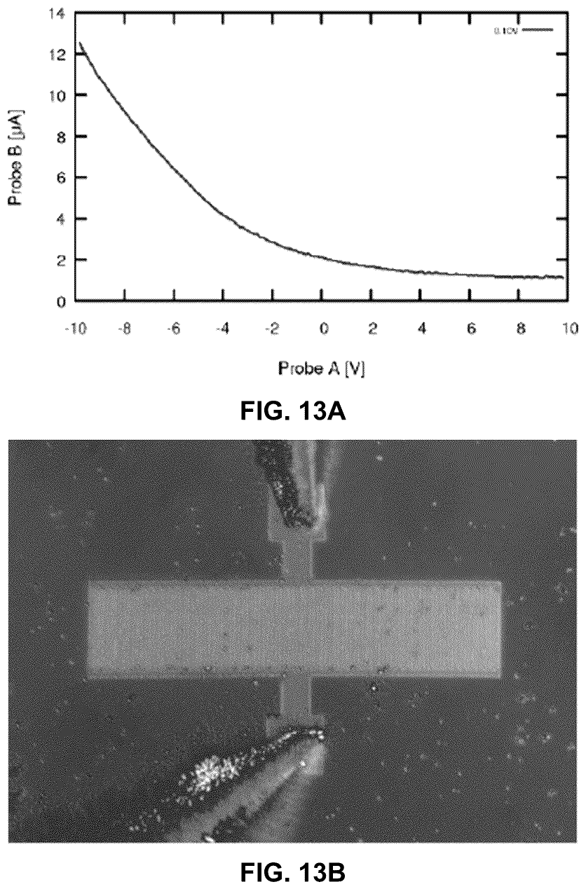

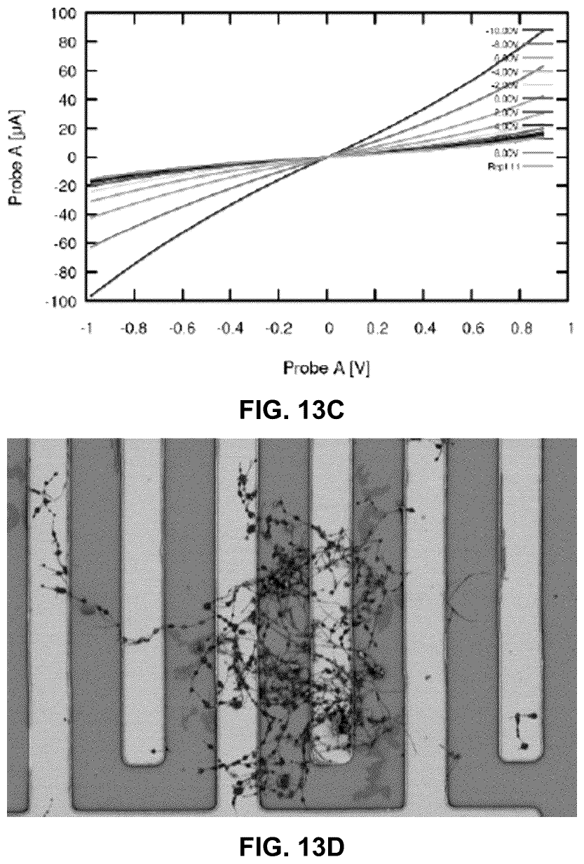

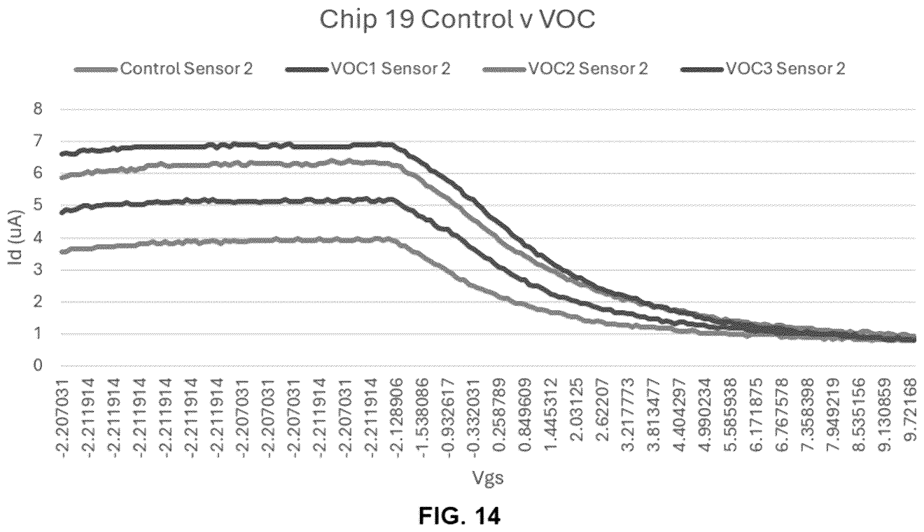

FIGS. 3A-3B and 13A-13D show how the CNT-FETs are tested. The CNT-FETs are tested using a 3-point probe and the parameters that will be measured include the 1) transconductance curve (I_{d} vs. V_{gs}) and 2) I_{d}-V_{ds} characteristics under different gate voltages. To ensure accuracy at low currents (10 μA range) and allow for customizable measurements, a source meter is fabricated based on the testing parameters. The required parameters include the following ranges: 1−I_{d}-V_{gs} curve) V_{ds}=−1V-1V (constant), V_{gs}=−10V-10V (sweep), and I_{d}=0-100 μA and 2-la-Vas curve) V_{ds}=−3V-3V (sweep), V_{gs}=−3V-3V (constant intervals), and I_{d}=0-10 μA.⁵⁰ The gate conductivity is also measured to ensure that the CNTs are semiconductive in nature. If the CNTs are conductive, the las will be in the 10⁻³ range and always in the “on” state, eliminating the ability to measure the shift in the I_{d}-V_{gs} curve for detection and CNT functionalization confirmation since the curve will be a horizontal line unlike the desired S-shaped curve which levels at Vth, further described below. To test conductivity, 2 probes are used with one acting as a ground and the other with a voltage sweep from −1V to 1V for a desired response in the 10⁻⁹. If the desired range is not achieved, a breakdown experiment will be conducted to breakdown walls of the CNTs to eliminate their conductive (metallic) components. This was done by fabricating a separate source meter module that includes the use of 2 probes and allows for-100V to 100V to be applied across the source-drain electrodes (source has voltage applied while drain is grounded) to decrease I_{d} from 550 μA to 10 μA. The voltage is applied incrementally for 28 second periods while monitoring the las so that when the current drops by 2 or more orders of magnitude, the voltage application is cut off. A program is included in the software of the data logger so that this process occurs automatically to avoid device burnout. Between each voltage application cycle, the I_{d}-V_{gs} curve and conductivity are measured until the desired ranges are achieved. Once semiconductivity of the CNTs has been achieved, the next step is for the horizontal shift in the I_{d}-V_{gs} curve to be measured at the optimal V_{gs} as determined by the I_{d}-V_{ds} curve measurements using the above initial (non-breakdown) source meter setup, by depositing non-functionalized CNTs on the FET surface and measuring the response then depositing functionalized CNTs on the surface of a different device to measure the shift in the I_{d}-V_{gs} curve to see the effect of adding various functional groups to the surface of the CNTs. Finally, one of the target compounds (for the specific functional group attached) is introduced to the functionalized CNT-FET to measure the next curve shift which corresponds to detection capability. This response is crucial in characterizing the device's ability to detect target pre-seizure VOCs. The discussed curve shifts for each stage can be seen in FIGS. 3A-3B. FIG. 14 shows VOC detection using exemplary sensors as disclosed herein.

### Example 2: Functionalization of Carbon Nanotubes for Recognition of Pre-Seizure Detection Sensor and Incorporation of Signal Processing

### Hydroxyl Functionalization

Hydroxyl groups were added to non-functionalized single-walled carbon nanotubes (SWCNTs) in order to prepare the SWCNTs for further functionalization. In some experiments, multi-walled carbon nanotubes (MWCNTs) were used to develop the functionalization protocol.

A glass reactor fitted with reflux condenser was filled with 250 ml of 0.5 M H₂SO₄. 5 g of KMnO₄ was added as oxidizing agent, and 2 g of MWCNTs were added. This dispersion was sonicated at 85° C. for 15 hours. The dispersion was filtered and MWCNTs were washed with concentrated HCL to remove MnO₂.

Surface analysis was conducted using transmission electron microscopy (TEM). The ETM sample was fabricated on a 300 mesh copper grid with a carbon film. TEM showed that oxidation of CNTs by a KMnO₄/H₂SO₄ mixture causes nanotube shortening, defect sites, and opened ends. Energy dispersive X-ray spectroscopy (EDS) showed an increase of oxygen content on the surface. Pristine CNTs had 2.8% surface oxygen, while functionalized CNTs showed 11.9% surface oxygen. EDS showed an increased oxygen: carbon ratio as well, with pristine CNTs showing a ratio of 0.03 (3%) while functionalized CNTs showed a ratio of 0.14 (14%).

### Carbon Nanotubes

Various carbon nanotubes can be used. SWCNTs and double-walled CNTs from Cheap Tubes, Inc. (Grafton, VT) were used in some experiments. These are OH-functionalized with an outer diameter of 1-4 nm, an inside diameter of 0.8-1.6 nm, ash <1.5 wt %, purity greater than 90 wt %. Additional MWCNT content was >5 wt % with amorphous carbon content of <3 wt %. Length is 0.5-2.0 μm and specific surface area of 407 m²/g. Electrical conductivity was >100 S/cm. This product contained 3.9% OH groups having a bulk density of 0.14 g/cm³ and a true density of about 2.1 g/cm³.

SWCNTs from Nanointegris (Boisbriand, QC, Canada) were also used. These had a diameter range of 1.2-1.7 nm, a length of from 300 nm to 5 μm, and a metal catalyst impurity of <1%. These further had an amorphous carbon impurity of 1-5% and electronic enrichment of 95%.

### KMnO₄ Optimization

100 mg pristine MWCNTs are dispersed in 20 mL of 5 M H₂SO₄ using sonication at room temperature for 30 min followed by vigorous stirring at 70° C. for 2 h to obtain a homogeneous mixture. Different amounts of KMnO₄ are added (0, 25, 50, 100, 200, and 250 mg) are added to prepare functionalized MWCNTs. Following stirring for 12 h at 70° C., these mixtures are cooled to room temperature and diluted with 200 ml of DI water. The product is separated from the suspension by centrifugation and thoroughly washed with DI water until neutral pH is achieved. The final obtained powders are dried under vacuum for 24 h at 70° C. Surface analysis was performed using various techniques as follows.

Field emission scanning electron microscopy (SEM) showed no sidewall damage. Oxidation treatment using 5 M H₂SO₄ and 25 mg KMnO₄ is an effective method of removing metallic catalysts from pristine MWCNTs without changing CNT length or shape. Larger concentrations of KMnO₄ begin to affect CNT structure.

EDS revealed changes in oxygen: carbon ratio after functionalization as shown in Table 1 below:

TABLE 1
Oxygen:Carbon Ratio after Functionalization

| Sample | O:C Ratio |
|---|---|
| Pristine | 9% |
| 25 mg KMnO₄ | 15.5% |
| 50 mg KMnO₄ | 21.83% |
| 100 mg KMnO₄ | 22.20% |
| 200 mg KMnO₄ | 25.74% |
| 250 mg KMnO₄ | 18.39% |

The increase in oxygen content indicates the CNTs underwent cleavage of C—C bonds, converting sp² hybridized carbon atoms to sp³ hybridized carbons to form hydroxyl and carboxylic functional groups.

UV-Vis spectroscopy was conducted on 1 mg of CNTs added to 10 mL of distilled water, followed by 60 min of sonication. All spectra were measured in 1 cm quartz cuvettes over 200 to 800 nm. An absorption peak is observed at 265 nm with no position shift for MWCNT samples. This spectroscopic feature is attributed to π→π′ transitions from C═C bonds. As expected, the absorbance value of functionalized MWCNTs increases with increased KMnO₄ amount.

FTIR was conducted to identify functional groups attached to the surface. An absorption peak at 3425 cm⁻¹ is attributed to stretching of hydroxyl groups and/or absorbed moisture on the nanotubes. The characteristic peak recorded at 1716 cm⁻¹ is due to C═O stretching and is found in all functionalized MWCNT samples. Characteristic bands at 1640 cm⁻¹ are likely due to stretching of carboxylic acids, indicating presence of COOH groups. Absorption bands at 1418 cm⁻¹ are unique to MWCNTs. Weak peaks at 2926 cm⁻¹ are assigned to an asymmetric CH stretching mode of CNT defects. Peaks at 700 cm⁻¹ and 1100 cm⁻¹ are ascribed to C—O bending and C—O stretching of the hydroxyl group.

Raman spectroscopy was carried out to identify structural changes of MWCNTs after functionalization. D and G bands are characteristic in graphite-like structures, with the intensity ratio of D and G bands compared to examine sidewall functionalization. There is a clear increase in Id/Ig when the amount of KMnO₄ increases (see Table 2):

TABLE 2
Id/Ig for Various Amounts of KMnO₄

| Sample | Id/Ig |
|---|---|
| Pristine | 1.29 |
| 25 mg KMnO₄ | 1.42 |
| 50 mg KMnO₄ | 1.48 |
| 100 mg KMnO₄ | 1.51 |
| 200 mg KMnO₄ | 1.60 |

The highest Id/Ig is recorded for the 200 mg KMnO₄ sample, which can be attributed to the highest degree of end opening of the nanotube tips. Despite this amount contributing to the greatest breakage of the CNT structure, functionalization of the OH groups is strongest. Tests were repeated for SWCNTs.

### APTES Functionalization

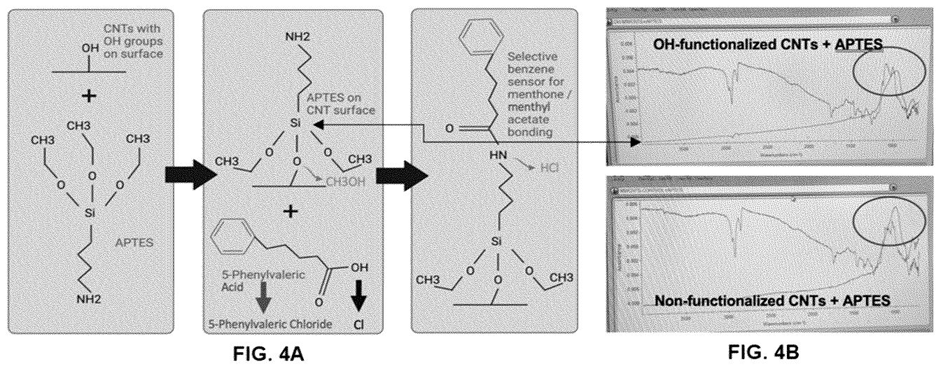

250 mg of functionalized CNTs were added to 100% ethanol and sonicated for 20 min. 500 mg APTES is added and refluxed at 100° C. with a magnetic stirrer. A reflux condenser is used to avoid solvent loss. The CNTs were centrifuged for 30 min at 22° C., 7000 rcf to form a pellet and ethanol and excess APTES are decanted. The pellet is resuspended in 15-20 ml of ethanol and vortexed. Centrifugation is repeated. The pellet is resuspended in 15-20 mL acetone, vortexed, and centrifuged. The pellet is resuspended in 15-20 mL of DI water, vortexed, and centrifuged. The sample was dried in a vacuum oven following the washing and centrifugation steps. Surface analysis is conducted as described above to confirm the addition of amine groups to the CNTs. EDS of APTES functionalized CNTs shows an increase in N and Si. See also FIGS. 4A-4B.

Functionalization of CNTs with Recognition Elements

Recognition elements that have been attached to immobilized CNTs are listed in Table 3 below:

TABLE 3
Recognition Elements for Pre-Seizure VOCs

| Head Group | Interaction Type | Target VOCs |
|---|---|---|
| 5-phenylvaleric | π-π bond with | menthone |
| acid/5- | benzene ring | L-menthyl acetate |
| phenylvaleric | | valencene |
| chloride | | β-cubebene |
| | | (—)-β-bourbene |
| Adipic acid/adipic | Polar bond with COOH | 4-tert-butylcyclohexyl acetate |
| coyl chloride | | camphor |
| | | (—)-β-bourbene |
| Pentadecanoic | Van der Waals interaction | pentadecanal |
| acid/pentadecenoyl | with long fatty chain | ethyl linalyl ether |
| chloride | | |

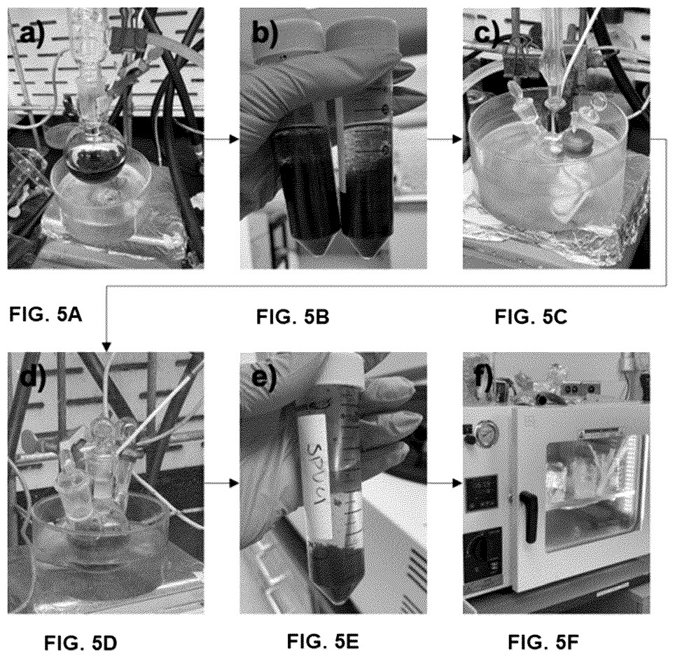

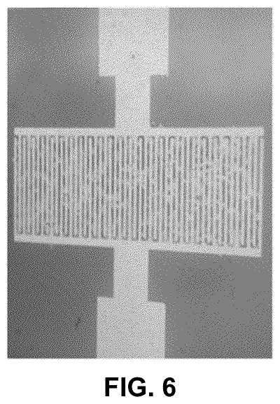

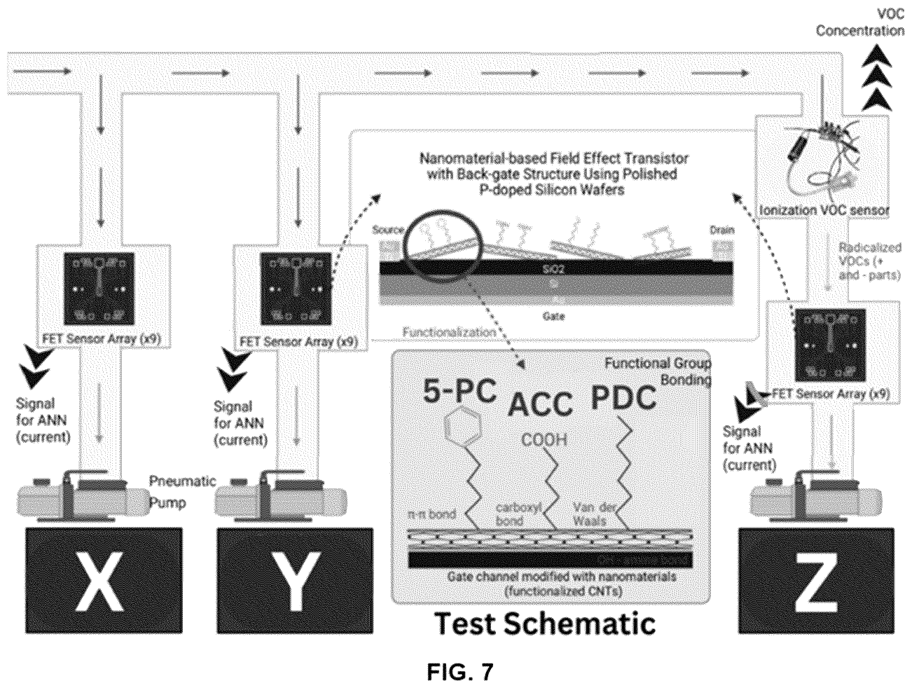

FIGS. 5A-5F show the CNT functionalization process, which begins by FIG. 5A: adding APTES to the OH-CNTs suspended in pure ethanol and placing the solution under reflux for 4 hours with a condenser to prevent solvent loss. Next, FIG. 5B: the APTES-CNTs are centrifuged to a pellet the excess ethanol is decanted to eliminate unbound APTES. Then the APTES-CNT pellet is resuspended in ethanol, acetone, and water with centrifuge/empty steps in between to ensure that the APTES-CNTs are thoroughly cleaned. The CNTs are then placed in the vacuum oven to dry back to their powder form. While drying, FIG. 5C: 5-phenylvaleric acid, adipic acid, and pentadecanoic acid are placed in separate flasks under nitrogen and vacuum as their conversion to chloride is a dry reaction. Once all air and moisture have left the flasks, thionyl chloride and a small volume of dried DMF are added to the 3 flasks. The adipic acid flask also has THE added to it because in the 5-PC and PDC reactions, a 5:1 ratio of thionyl chloride:acid is used so the thionyl chloride acts as both the reactant and solvent, but for the adipic acid, a 1:1 ratio is used to prevent having an excess since the goal is to chloridate just one of the OH groups per molecule. Then, the solutions are again placed under reflux for 4-5 hours before being connected to a short path distillation apparatus under vacuum for 15-20 minutes to remove excess thionyl chloride. Once completed, the acids should now be chlorides, and FIG. 5D: the APTES-CNTs are added to 3 additional separate flasks and placed under vacuum and nitrogen before adding dried DMF and each acyl chloride (5-PC, PDC, and ACC). Then, the solution is placed back into the bath for 24 hours at 100° C. After the reaction is complete, the flask is opened to quench the unreacted acyl chlorides, and FIG. 5E: the centrifuge/empty/resuspend cycle is repeated using DI water then THF 3 times to remove excess solvent from the functionalized CNTs. Then, FIG. 5F: each of the CNTs are placed in the vacuum oven to dry and are ready for application on the CNT-FET surface. Specific process steps for each recognition element are detailed below.

Phenylvaleric chloride: Add 0.89 g of 5-phenylvaleric acid to a 3-neck round bottom flask. Place flask in silicone oil bath and connect to nitrogen line and vacuum line. Once air and moisture have been evacuated, add 1.45 mL of thionyl chloride and 100 μL of dry DMF to the flask. Reflux at 90° C. for 14 h. Remove excess thionyl chloride using short path distillation under vacuum.

Add 50 mg of APTES-CNTs to flask. Place under vacuum followed by nitrogen. Add 50 mL of dry DMF to flask. Add 250 mg 5-phenylvaleric chloride to flask. Incubate for 24 h at 100° C. under vacuum/nitrogen with magnetic stirring. Open flask to expose to air and transfer to a beaker; unreacted 5-phenylvaleric chloride will be quenched to form HCl. Add water to the functionalized CNTs for further quenching. Centrifuge for 20 min at 22° C. and 7000 rcf. Resuspend pellet in DI water. Vortex in THF; centrifuge and resuspend 3 times (20 minutes, 22° C., 7000 rcf) and dry CNTs in vacuum oven.

Pentadecanoyl chloride: A similar procedure was used as for phenylvaleric chloride to functionalize APTES-CNTs.

Adipic coyl chloride: The procedure used for phenylvaleric chloride and pentadecenoyl chloride was modified to account for multiple COOH groups present in adipic acid. 444 mg of adipic acid were added to a flask under vacuum and nitrogen. 10 mL dry THF were added to the flask. 360 mg of thionyl chloride (1:1 mole ratio with adipic acid) are added along with 1 drop of dry DMF as catalyst. A reflux condenser is added to prevent the loss of THF and thionyl chloride. Reflux is conducted at 66° C. for 3 h. Following reaction, THF is removed under a 2-stage short path distillation lasting for 20 min (first under ambient pressure, then under vacuum).

Add dry DMF and 1 mg APTES CNTs to flask along with adipic coyl chloride. Incubate for 24 h at 100° C. under vacuum/nitrogen with magnetic stirring. Open flask to expose to air and transfer to a beaker. Add water to the functionalized CNTs for further quenching. Centrifuge for 20 min at 22° C. and 7000 rcf. Resuspend pellet in DI water. Vortex in THF; centrifuge and resuspend 3 times (20 minutes, 22° C., 7000 rcf) and dry CNTs in vacuum oven.

Analysis: FTIR characterization of functionalization of CNTs with phenylvaleric chloride (5-PC), pentadecenoyl chloride (PDC), and adipic coyl chloride (ACC) was assessed using FTIR with KBr pellet. EDS characterization of 5-PC, ACC, and PDC showed an increase of chloride.

### Example 3: Testing and Validation

Testing begins with FTIR and zeta potential (ZP) measurements to test the functionalization reactions taking place. The SWCNTs with OH-surface groups serve as a control parameter, measured using FTIR/ZP. Next, APTES was applied to the CNTs, and FTIR/ZP will be used again to confirm the covalent bond formed. Then, a control of the acid groups was taken using FTIR/ZP prior to/after applying thionyl chloride and dried DMF for bonding confirmation. Finally, the acyl chloride groups were bonded to the APTES, and FTIR/ZP was again used for attachment confirmation. After the CNT-FETs were functionalized with their recognition elements, they were introduced to one of the target VOCs for bonding confirmation prior to placement in the functionalized FET arrays. Scanning electron microscopy (SEM) was used to identify the location of deposited functionalized CNTs on the gate channel between the source and drain electrodes. SEM was also used after applying the inner layer for common source and drain establishment, to ensure that at least 1/16 sensors on each chip have CNTs within their gate channel. XPS, NMR, and Raman Spectroscopy are other methods that can be used for characterization, along with electrical characterization.

Once the three different channels in the model were established (nonfunctionalized FET, functionalized FET, and ionized FET), four signal parameters will be collected for each VOC and the phantom solution matrix creation began by introducing target mixtures to the setup: VOC 1, 1-2, 1-3, 1-4, 2, 2-3, 2-4, etc. By collecting the parameters for these arrays, the sensor develops a fingerprint for each biomarker. This allows for the first use of machine learning for the detection of this profile. Once the algorithm was established and the sensor taught, it was ready for testing. First, the 9-VOC phantom solution will be tested with seizure alert dogs to confirm seizure association before completing the microcontroller learning. Then, the sensor will be tested in a controlled environment through direct gas insertion of the final target analyte solution to confirm that the fabricated schematic will accurately report a seizure and indicate no seizure presence when alternative gas mixtures are introduced.

### Glass Substrate Experiment to Confirm APTES Bonding to CNTs and Acyl Chloride Groups to APTES

APTES is used as a baseline linker to CNTs and acyl chloride groups will be applied to serve as selective linkers for non-covalent binding to the target VOCs. Prior to addition, 5-phenylvaleric acid is reacted with thionyl chloride as shown in Scheme 1:

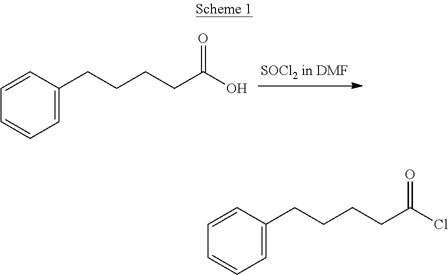 [SMILES: O=C(O)CCCCc1ccccc1 ; O=C(Cl)CCCCc1ccccc1]

Functionalization proceeds in two steps as follows. 3 mg of CNTs are added to 3 mL of DMF and sonicated for one hour. 5 μL of APTES in 1.95 mL of ethanol are mixed and added to the CNT/DMF solution. Separately, 5-phenylvaleric acid in DMF is reacted with thionyl chloride. The CNT/DMF/APTES/ethanol solution and the 5-phenylvaleric acid/DMF/thionyl chloride solutions are mixed and applied to the FET after outer layer deposition as described above.

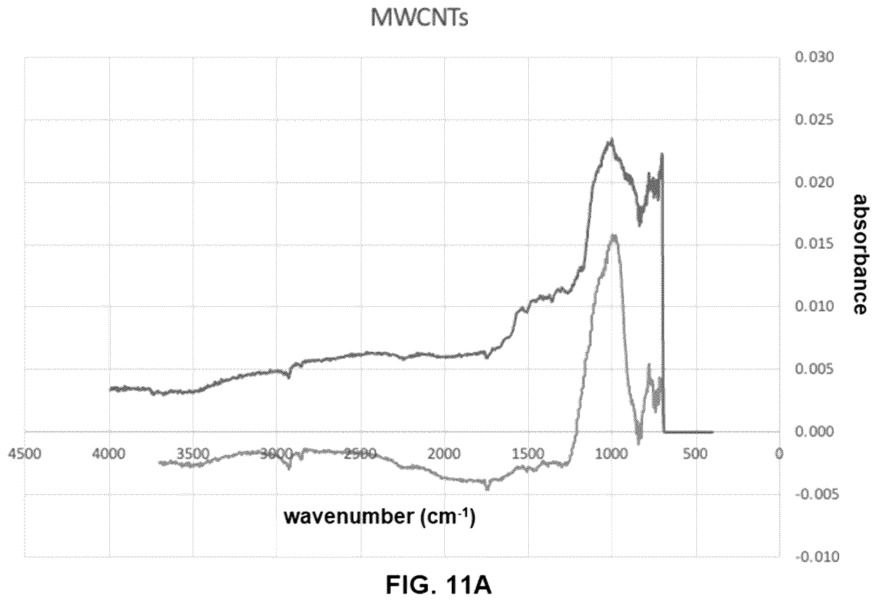

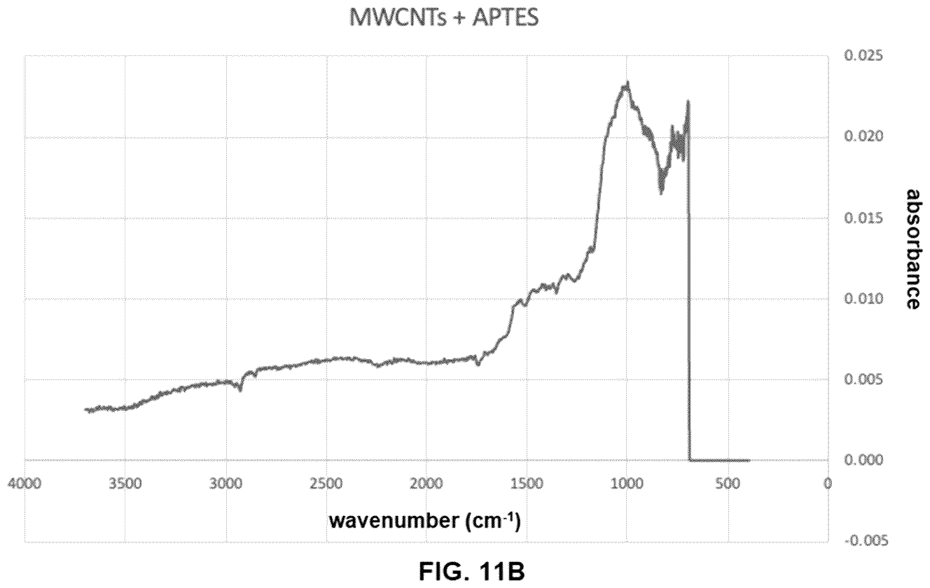

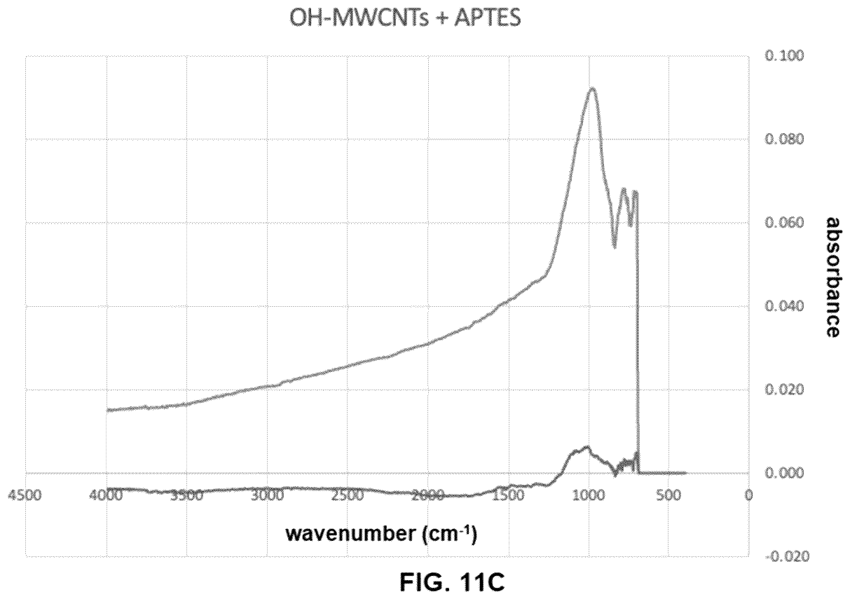

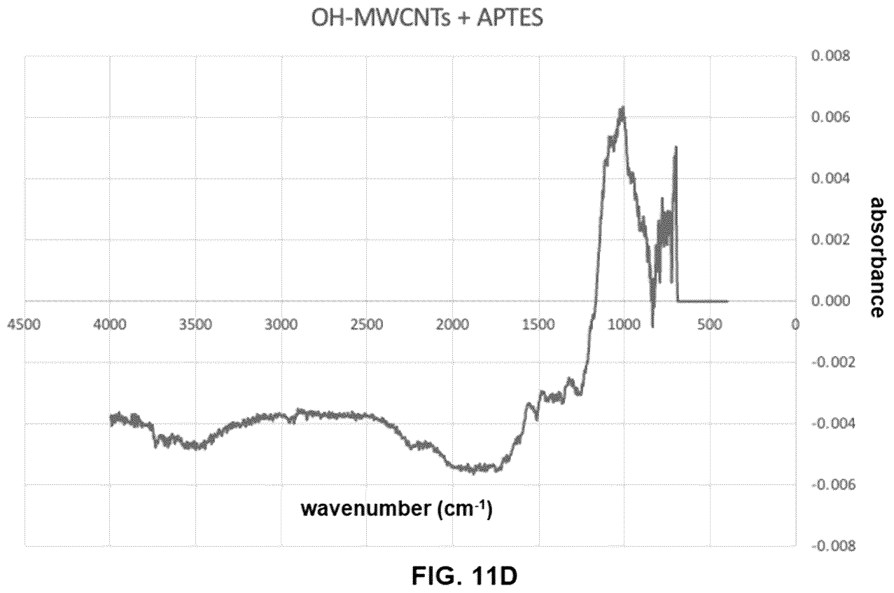

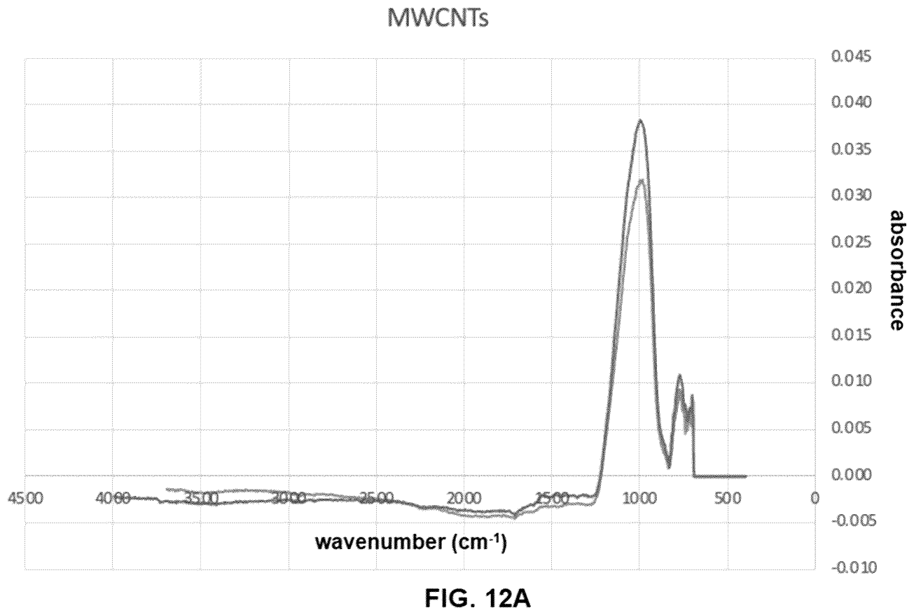

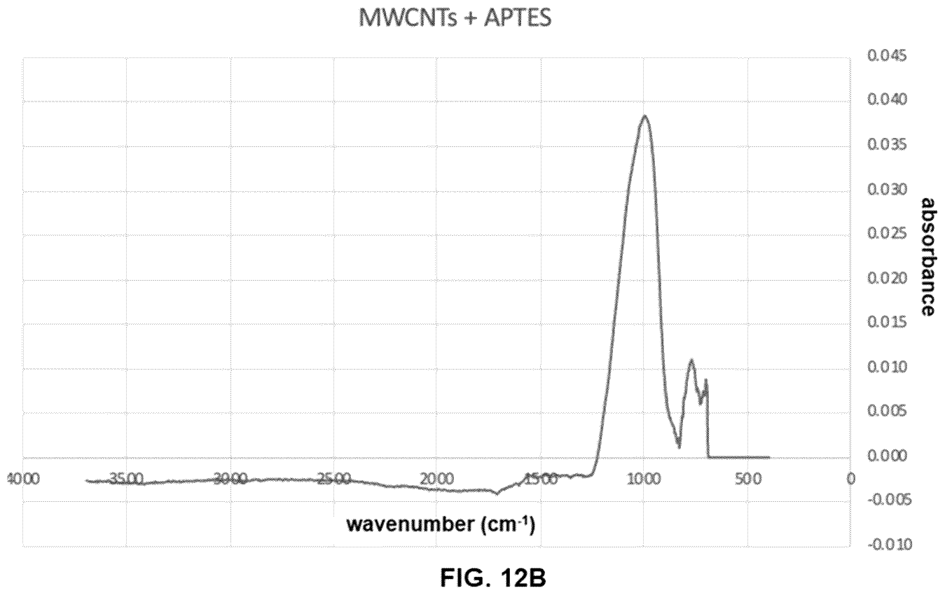

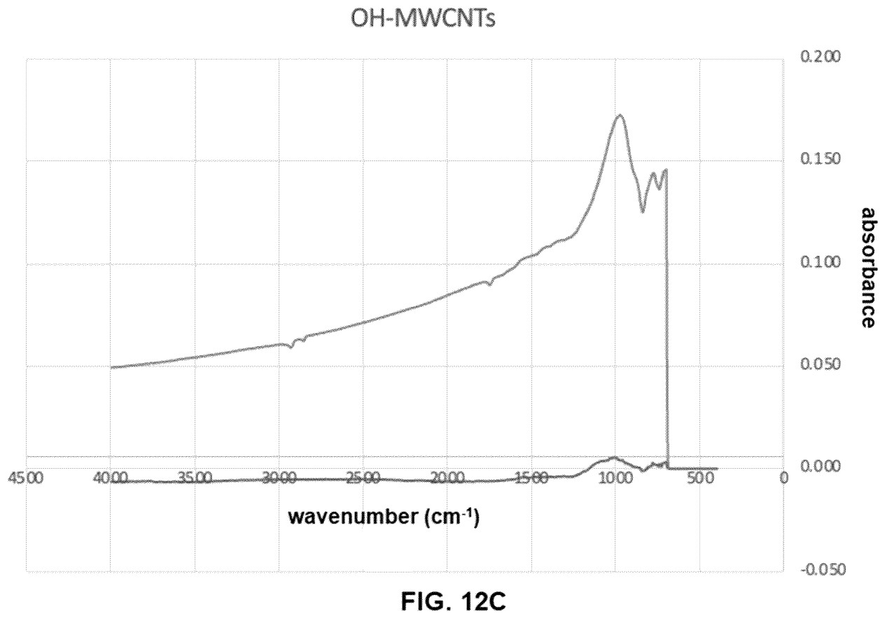

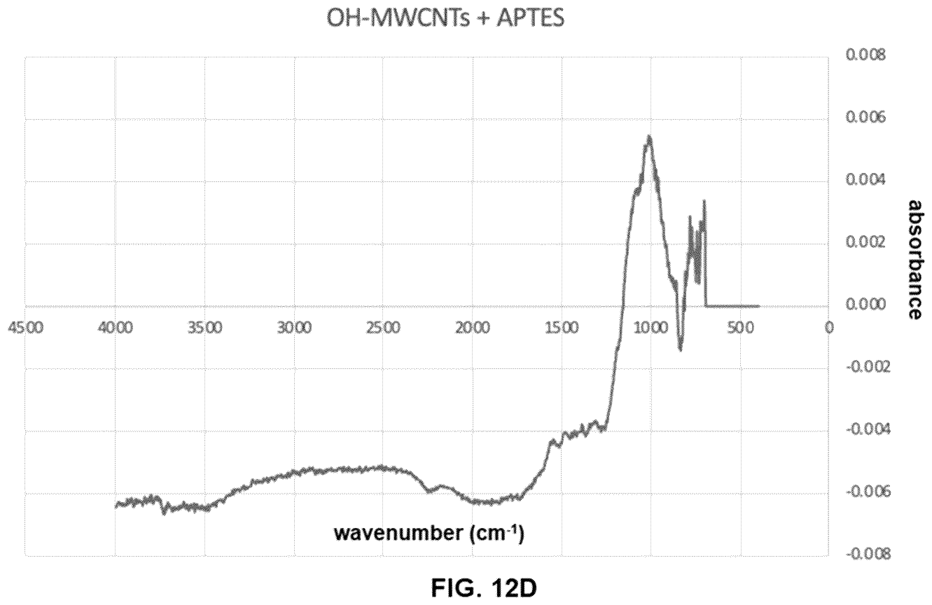

APTES functionality can be tested as follows. APTES is mixed with ethanol in a 1:1 volume ratio. A glass substrate is cleaned and placed on copper. A plasma arc is run over the glass for 1 min and the 100 μL of the APTES and ethanol solution is applied to the glass. After 30 s, a fluorescein NHS ester solution is applied to the glass and allowed to react for 5 min The glass is then washed and checked for fluorescence. In some experiments, CNTs are applied to a glass slide and allowed to dry for 1 h. 46.84 μL APTES is mixed with 19.9953 mL ethanol and applied to CNTs on a glass slide. The APTES: ethanol solution is applied, followed by fluorescein NHS ester, and fluorescence and/or FTIR is used to monitor for the presence of fluorescein after several washes. Exemplary FTIR spectra for the samples and controls are shown in FIGS. 11A-12D.

### Signal Processing

The disclosed sensors can be connected to equipment for processing detection of the pre-seizure VOCs including, but not limited to, a photoionization detector, a transducer and/or other means for converting a received signal, an amplifier, a microprocessor, and a computer or other display device. In any of these modules, the levels of analyte detected can be compared to a reference sample in order to avoid false positives.

In some embodiments, the disclosed sensors can be integrated into devices that collect other vital signs from the subject wearing them including heart rate, body temperature, skin impedance, and movements are associated with various types of seizures. Computation units or modules of the devices can incorporate an artificial neural network in order to process the signals received. It is expected the devices will have to undergo a training period to differentiate different types of seizures in a given individual; however, the devices should work immediately for general seizure prediction even for a new user. Detection of the VOCs by the device will trigger the vital sensors to begin monitoring.

The source-drain current will be tested at different gate voltages to observe response at control and for different VOCs. Parameters will be collected for several VOC/phantom mixtures to train the ANN to recognize each of the target VOCs. Output from the vital monitors will be integrated into the ANN so that detection of seizure presence and type of seizure can be integrated.

In the disclosed devices, sensing is based on monitoring the source-drain currents, which change based on interaction of the functionalized CNTs with specific VOCs. Control or nonfunctionalized sensor readings will be compared to readings from sensors functionalized with recognition elements for specific VOCs will be compared to determine the presence of VOCs. The Id-Vgs curve should shift to the right upon detection.

### Sensor Training

Each pre-seizure VOC will be individually introduced to a series of connected gas columns that will collect four parameters to create a fingerprint for each VOC. The first column will include the nonfunctionalized array of CNT-FETs for a control to compare to the second column, which includes an array of functionalized CNT-FETs. The third column contains a commercial VOC sensor with fabricated ionization chamber to compare current level to concentration. This third column compares current level to concentration to determine the concentration of each pre-seizure VOC emitted from an individual subject prior to an episode. The fourth column is a second array of functionalized CNT-FETs collect information about the ionized part of each VOC once it has traveled through the VOC sensor.

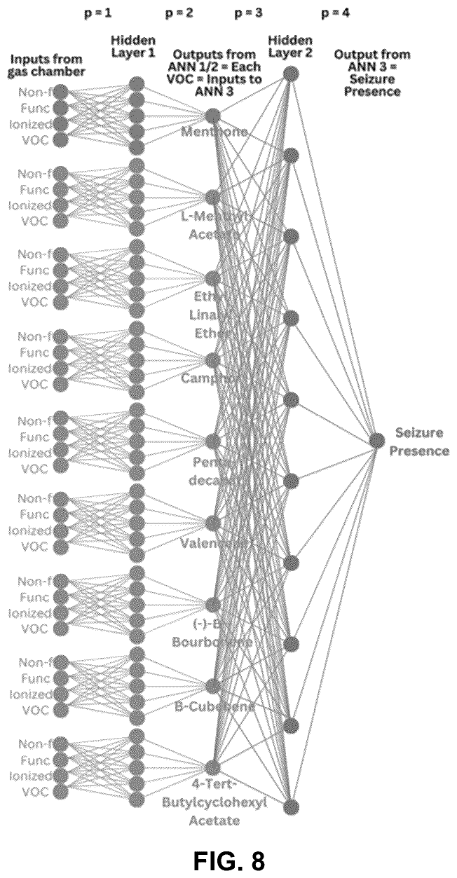

Training is conducted using artificial neural networks (ANN); see FIG. 8. ANN1 takes 4 inputs from gas column arrays for one VOC. The arrays are non-functionalized (OH-CNTs) for control and CNTs functionalized with ACC, 5-PC, and PDC to measure change from control, ionized, and VOC for concentration level of VOCs. The output of ANN1 is 1/9 of the target VOCs. ANN2 implements the same network used for a single VOC to all nine VOCs. Each VOC has its own set of 4 inputs from the gas columns as with AN1 and the output is all 9 of the target gases. The network can selectively identify which of the 9 gases are present based on inputs. ANN3 takes outputs from ANN2 and uses them as inputs. The output of ANN3 is seizure presence by examining the level of each of the 9 VOCs present from the epileptic individual. This network is trained using test subjects, but ANN1 and ANN2 can begin training using gas column arrays.

The sensor is taught in vitro by measuring these phantom VOC mixtures in arrays (seizure) and comparing the resulting signals to other perfumes (not seizure). The target gases (9 pre-seizure VOCs) will be fed into the column marked by the arrow on the left. The test setup is created in such a way that each VOC has its own integrated string of 3 sensors that are taught to detect only one target through the input of 4 resultant parameters. The four parameters collected for each VOC include: control source-drain current (I_{DS}) signal through an unfunctionalized CNT-FET, target I_{DS} signal through a functionalized CNT-FET, ionized I_{DS} signal through a functionalized CNT-FET following radicalization from a photoionization detector (PID), and a concentration value from the PID through the electrons and positive ions measured as a current proportional to the concentration of the compound. The first column that the selected gas(es) flow through generates the control signal by measuring the source-drain current (I_{DS}) through an unfunctionalized CNT-FET. In other words, this control sensor array will not have the recognition elements attached to the OH-CNTs that are presented the disclosed sensor array. This allows the artificial neural network (ANN) to have a base signal to compare to the functionalized array in column 2 for each VOC that the microcontroller is learning. This second column that the gas(es) flow through has CNT-FETs with recognition elements attached to the surface CNTs, which will serve as the target signal by measuring the I_{DS} through the sensors in this array. The sensors in this column are shown in between the second and third columns; the CNT-FET structure is pictured on the top with functionalized CNTs which are shown with further detail directly below. The final signals that are fed to the ANN are found in the third column, the ionized group. The gas(es) will first flow through a photoionization detector (PID) sensor to determine the concentration level of each of the target VOCs (current is proportional to concentration), then the ionized parts will flow through a second functionalized CNT-FET array, collecting the I_{DS} of the more prominent radicalized part for a 4th parameter to feed into the ANN. The PID sensor is housed inside of an ionization chamber machined to be airtight and allow tubes to be connected to the inlet with a pneumatic pump at the outlet to force test gases through. Each parameter is collected from one of three gas columns that consist of an array with the associated sensors indicated for detection. Overall, the ANN has 4 parameters for each of the target VOCs, and it will be taught through phantom mixtures of VOCs by introducing 1-9 at a time until all of them have identified values.

The curve shifting shown in FIG. 14 was collected using a gas flow setup wherein VOCs are introduced through deposition on an open syringe; air is pulled through tubing to a silicone gas chamber using a pneumatic pump; a silicone gas chamber holds the chip wire bonded to a PCB for external connection and gas flow testing. The VOCs are introduced to the input apparatus through deposition onto a lint-free cloth; the excess sample falls to the collection chamber below. The VOCs travel through PTFE tubing and can be halted using a manual luer-lok valve. Once surpassing the valve, the target VOCs travel through a gas flow chamber with two-air tight compartments to ensure sufficient sample flow over the sensor's surface for detection. A mass flow controller is used to control the mass flow rate and a pneumatic pump is used to allow the VOCs to travel through the chamber.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Bruno, E. et al., “Seizure Detection at Home: Do Devices on the Market Match the Needs of People Living with Epilepsy and Their Caregivers?,” Epilepsia, 2020, 61: S11-S24.
2. Catala, A., et al. Dogs demonstrate the existence of an epileptic seizure odour in humans. Sci Rep, 2019, 9, 4103.
3. Davis, P. R. N. “The Investigation of Human Scent from Epileptic Patients for the Identification of a Biomarker for Epileptic Seizures” 2017, Dissertation: Doctor of Philosophy Chemistry, Florida International University.
4. Devinsky, O. et al., “Epilepsy,” Nature Reviews Disease Primers, 2018, 4.
5. Doroshenko, I. et al. Infrared Absorption Spectra of Monohydric Alcohols. Dataset Papers in Science. 2013:329406.
6. Fisher, R S, et al. ILAE official report: a practical clinical definition of epilepsy. Epilepsia. 2014, 55:475-82.
7. Howland, R. H. “Vagus Nerve Stimulation,” Current Behavioral Neuroscience Reports, 2014 1:64-73.
8. Khatib, M. et al, “Sensors for Volatile Organic Compounds,” ACS Nano, 2022, 16:7080-7115.
9. Liang, Y. et al., “Wafer-Scale Uniform Carbon Nanotube Transistors for Ultrasensitive and Label-Free Detection of Disease Biomarkers,” ACS Nano, 2020:8866-74.
10. Maa, E., et al. Canine detection of volatile organic compounds unique to human epileptic seizure. Epilepsy & Behavior, 2021, 115, 107690.
11. Olsson, M. J. et al., “The Scent of Disease: Human Body Odor Contains an Early Chemosensory Cue of Sickness,” Psychological Science, 2014, 25:817-23.
12. Onorati, F. et al., “Prospective Study of a Multimodal Convulsive Seizure Detection Wearable System on Pediatric and Adult Patients in the Epilepsy Monitoring Unit,” Frontiers in Neurology, 2021, 12:724904.
13. Shirasu, M. et al, The scent of disease: volatile organic compounds of the human body related to disease and disorder, 2011, The Journal of Biochemistry, 150:257-266.
14. Sperling, M. The Consequences of Uncontrolled Epilepsy. CNS Spectrums, 2004, 9:98-109.
15. Stafstrom C. E. et al, “Seizures and Epilepsy: An Overview for Neuroscientists,” Cold Spring Harbor Perspectives in Medicine, 2015, 5: a022426-a022426.
16. Strong, V. et al., Seizure-alert dogs—fact or fiction?, Seizure, 1999, 8:62-65, ISSN 1059-1311.
17. Ulate-Campos, A. et al., Automated seizure detection systems and their effectiveness for each type of seizure, Seizure, Volume 2016, 40:88-101.
18. Wang, B. et al., “Artificial Sensing Intelligence with Silicon Nanowires for Ultraselective Detection in the Gas Phase,” Nano Letters, 2014:933-38.
19. Zamzami, M. A. et al, Fabrication and characterization of field effect transistor based on single walled carbon nanotubes, Journal of King Saud University-Science, 2022, 34:102137.
20. Zamzami, M A, et al. Carbon nanotube field-effect transistor (CNT-FET)-based biosensor for rapid detection of SARS-COV-2 (COVID-19) surface spike protein S1. Bioelectrochemistry, 2022, 143:107982.

What is claimed is:

1. A sensor for predicting epileptic seizures in a subject, the sensor comprising a chip comprising a layer of carbon nanotubes (CNTs),
wherein the CNTs are functionalized to comprise a reactive species that is chemically bonded to at least one recognition element;
wherein the at least one recognition element non-covalently binds with one or more pre-seizure volatile organic compounds (VOCs).

2. The sensor of claim 1, wherein the pre-seizure VOCs are emitted by the subject in saliva, sweat, breath, or any combination thereof, and wherein the sensor is worn by the subject on or near a body surface that emits saliva, sweat, breath, or any combination thereof.

3. The sensor of claim 2, wherein the body surface comprises the wrist, underarm, arm, neck, or another skin surface.

4. The sensor of claim 2, wherein the sensor is, or is a component of, a smart watch, armband, necklace, or skin patch.

5. The sensor of claim 1, wherein the CNTs are functionalized with (3-aminopropyl) triethyoxysilane (APTES).

6. The sensor of claim 1, wherein the chip comprises a discontinuous SiO₂ layer comprising exposed areas of silicon and areas of SiO₂.

7. The sensor of claim 6, further comprising at least one metal in contact with at least a portion the exposed areas of silicon.

8. The sensor of claim 7, wherein the at least one metal comprises Cr, Au, or any combination thereof.

9. The sensor of claim 6, wherein the CNTs are deposited on the exposed areas of SiO₂.

10. The sensor of claim 1, wherein the one or more pre-seizure VOCs comprise menthone, L-menthyl acetate, valencene, β-cubebene, (−)-β-bourbene, 4-tert-butylcyclohexyl acetate, camphor, pentadecanal, ethyl linalyl ether, or any combination thereof.

11. The sensor of claim 1, wherein the at least one recognition element comprises 5-phenylvaleric chloride, adipic coyl chloride, pentadecanoyl chloride, or any combination thereof.

12. The sensor of claim 11, wherein the at least one recognition element is 5-phenylvaleric chloride and interacts with the one or more pre-seizure VOCs via IT-TT bonding with an aromatic ring in the 5-phenylvaleric chloride, wherein the one or more pre-seizure VOCs comprise menthone, L-menthyl acetate, valencene, β-cubebene, (−)-β-bourbene, or any combination thereof.

13. The sensor of claim 11, wherein the at least one recognition element is adipic coyl chloride and interacts with the one or more pre-seizure VOCs via polar bonding with a carboxylic acid group in the adipic coyl chloride, wherein the one or more pre-seizure VOCs comprise 4-tert-butylcyclohexyl acetate, camphor, (−)-β-bourbene, or any combination thereof.

14. The sensor of claim 11, wherein the at least one recognition element is pentadecanoyl chloride and interacts with the one or more pre-seizure VOCs via van der Waals interactions with a long fatty chain in the pentadecanoyl chloride, wherein the one or more pre-seizure VOCs comprise pentadecanal, ethyl linalyl ether, or any combination thereof.

15. The sensor of claim 1, wherein the carbon nanotubes comprise single walled carbon nanotubes (SWCNTs), multi walled carbon nanotubes (MWCNTs), or any combination thereof.

16. A method of predicting an epileptic seizure in a subject, the method comprising using the sensor of claim 1 to sense emission by the subject of one or more pre-seizure VOCs.

17. The method of claim 16, wherein the epileptic seizure is predicted from about 10 minutes to about 45 minutes prior to occurring.

18. The method of claim 16, wherein the method further comprises collecting at least one vital sign from the subject, wherein the at least one vital sign is correlated to epileptic seizure type.

19. The method of claim 18, wherein the at least one vital sign comprises heart rate, body temperature, skin impedance, body movements, or any combination thereof.

20. The method of claim 18, wherein the epileptic seizure type comprises an absence seizure, a tonic-clonic seizure, a simple focal seizure, a complex focal seizure, a secondary generalized seizure, or any combination thereof.
